(19) United States
(12) Patent Application Publication
(10) Pub. No.: US 2004/0092568 A1
(43) Pub. Date: May 13, 2004

(54) Methods for the treatment, prevention and management of macular degeneration

(76) Inventor: Jerome B. Zeldis, Princeton, NJ (US)

Correspondence Address: JONES DAY, 222 EAST 41ST STREET, NEW YORK, NY, 10017, US

(21) Appl. No.: 10/699,105

(22) Filed: Oct. 30, 2003

Related U.S. Application Data
(60) Provisional application No. 60/422,896, filed on Oct. 31, 2002.

(51) Int. Cl. A61K 31/416

(52) U.S. Cl. 514/406

(57) ABSTRACT

The present invention relates to methods for treating, preventing and/or managing macular degeneration (MD). Specific embodiments encompass the administration of a JNK Inhibitor, alone or in combination with a second active agent and/or surgery or physical therapy. Pharmaceutical compositions, single unit dosage forms, and kits suitable for use in methods of the invention are also disclosed.

This application claims the benefit of U.S. provisional application No. 60/422,896, filed Oct. 31, 2002, the contents of which are incorporated by reference herein in their entirety.

### FIELD OF INVENTION

This invention relates to methods for treating, preventing and/or managing macular degeneration (MD) and related syndromes, which comprise the administration of a JNK Inhibitor alone or in combination with a known therapeutic. The invention also relates to pharmaceutical compositions and dosing regimens. In particular, the invention encompasses the use of a JNK Inhibitor in conjunction with surgical intervention, and/or another standard therapy for macular degeneration.

BACKGROUND OF THE INVENTION

Pathobiology of Macular Degeneration

Macular degeneration (MD), which is also referred to as age-related macular degeneration (AMD), is an eye disease that destroys central vision by damaging the macula. The macula is part of the retina, a thin layer of nerve cells that lines most of the inside of the eyeball. The nerve cells in the retina detect light and send to the brain signals about what the eye sees. The macula is near the center of the retina at the back of the eyeball and provides the clear, sharp central vision that an animal uses for focusing on what is in front of it. The rest of the retina provides side (peripheral) vision.

There are two forms of MD: exudative (wet) and atrophic (dry). Riordan-Eva, P., Eye, in Current Medical Diagnosis and Treatment, 41 ed. 210-211 (2002). Ninety percent of patients have the dry form, while only ten percent have the wet form. However, patients with the wet form can lose up to ninety percent of their vision. DuBosar, R., J. of Ophthalmic Nursing and Technology, 18: 60-64 (1998).

Macular degeneration results in the presence of choroidal neovascularisation (CNVM) and/or geographic atrophy of retinal pigment epithelium (RPE) in an eye with drusen. Bird, A. C., Surv. Ophthamol. 39:367-74 (1995). Drusen are rounded whitish-yellowish spots in the fundus, located external to the neuroretina. Additional symptoms of MD include RPE detachment (PED) and submacular disciform scar tissue. Algvere, P. V., Acta Ophthalmologica Scandinavica 80:136-143 (2002).

Choroidal neovascularisation is a problem that is related to a wide variety of retinal diseases, but is most commonly associated with MD. CNVM is characterized by abnormal blood vessels stemming from the choroid (the blood vessel-rich tissue layer just beneath the retina) growing up through the retinal layers. These new vessels are very fragile and break easily, causing blood and fluid to pool within the layers of the retina. As the vessels leak, they disturb the delicate retinal tissue, causing the vision to deteriorate. The severity of the symptoms depends on the size of the CNVM and its proximity to the macula. Patients' symptoms may be very mild, such as a blurry or distorted area of vision, or more severe, such as a central blind spot.

Patients having drusen and possibly pigmentary abnormalities, but no CNVM or geographic atrophy, are generally diagnosed as having age-related maculopathy (ARM). Id. The histopathological hallmark of ARM and MD is a continuous layer of fine granular material deposited in the inner part of Bruch's membrane at the base of the RPE cells. Sarks, J. P., et al., Eye 2(Pt. 5):552-77 (1988). These basal deposits are though to be accumulated as waste products from the continuing RPE phagocytosis or photoreceptor outer segment material. The basal deposits lead to a thickening and decreased permeability of Bruch's membrane. It has been hypothesized that decreased water permeability impairs an exchange of nutrients, traps water and enhances the development of soft drusen and PED and eventually leads to atrophy of RPE cells. Id. However, the current overall understanding of ARM and MD pathogenesis is incomplete. Cour, M., et al., Drugs Aging 19:101-133 (2002).

Because MD is most prevalent in the elderly, the fastest growing segment of the population, MD is destined to become a major problem economically and socially. Macular degeneration is the most common cause of visual loss in developed countries in individuals over the age of 60. Macular degeneration has obliterated the central vision of 1.7 million Americans and another 11 million are at risk. DuBosar, R., J. of Ophthalmic Nursing and Technology, 18: 60-64 (1998). Currently, there is no known cure. Rhoodhooft, J., Bull. Soc. belge Ophtalmol. 276:83-92 (2000). Thus, there is an urgent need for effective treatments for MD.

Treatment of Macular Degeneration

Until recently, laser photocoagulation was the only treatment routinely used for MD, and it provides only modest results. Laser photocoagulation is a type of laser surgery that uses an intense beam of light to burn small areas of the retina and the abnormal blood vessels beneath the macula. The burns form scar tissue and seal the blood vessels, keeping them from leaking under the macula. Laser photocoagulation is effective only for patients having wet MD. Furthermore, laser photocoagulation is a viable option for only about 13% of those patients. Joffe, L. et al., International Ophthalmology Clinics 36(2): 99-116 (1996). Laser photocoagulation does not cure wet MD, rather it sometimes slows down or prevents further loss of central vision. Without treatment, however, vision loss from wet MD may progress until a person has no remaining central vision.

The most serious drawback to laser surgery is that the laser damages some of the nerve cells in the macula that react to light, causing some vision loss. Sometimes, the vision loss resulting from surgery is as severe or worse than the vision loss resulting from no treatment. In some patients, however, laser surgery initially worsens vision, but prevents more severe loss of vision over time.

Verteporfin has recently been used to treat wet MD. Cour, M., et al., Drugs Aging 19:101-133 (2002). Verteporfin is a blood-vessel-blocking photoreactive dye that is administered via injection. The dye moves to the blood vessels that are responsible for the loss of sight and is then activated by shining a non-burning beam of light into the eye in the presence of oxygen. Verteporfin is transported in the plasma primarily by lipoproteins. Activated verteporfin generates highly reactive, short-lived singlet oxygen and reactive oxygen radicals, resulting in local damage to neovascular endothelium. This causes vessel occlusion. Damaged endothelium is known to release procoagulant and vasoactive factors through the lipo-oxygenase (leukotriene) and cyclo-oxygenase (eicosanoids such as thromboxane) pathways, resulting in platelet aggregation, fibrin clot formation and vasoconstriction. Verteporfin appears to somewhat preferentially accumulate in neovasculature, including choroidal neocovasculature. However, animal models indicate that verteporfin also accumulates in the retina. Therefore, verteporfin administration might collaterally damage retinal structures, including the retinal pigmented epithelium and outer nuclear layer of the retina.

Another strategy currently being investigated for the treatment of MD is pharmacological antiangiogenic therapy. Cour, M., et al., Drugs Aging 19:101-133 (2002). However, a first clinical trial with an antiangiogenic agent, interferon-a, showed that it was ineffective at treating MD and resulted in a high rate of adverse effects. Arch. Ophthalmol. 115:865-72 (1997).

Intravitreal injection of triamcinolone reportedly inhibits the growth of laser-induced CNVM in monkeys, but fails to prevent severe visual loss over a one-year period in patients with MD in a randomized trial. Gillies, M. C., et al., Invest. Ophthalmol. Vis. Sci. 42:S522 (2001). A number of other antiangiogenic drugs are in various stages of development for use in patients with MD, including angiostatic steroids (e.g., anecortave acetate, Alcon) and vascular epidermal growth factor (VEGF) antibodies or fragments thereof. Guyer, D. R., et al., Invest. Ophthalmol. Vis. Sci. 42:S522 (2001). One such VEGF antibody is rhuFab. Additional new drugs for the treatment of MD include EYE101 (Eyetech Pharmaceuticals), LY333531 (Eli Lilly), Miravant and RETISERT implant (Bausch & Lomb), which exudes a steroid into the eye for up to three years.

Although new and promising strategies for the treatment of MD and related macular degenerative diseases are being investigated, there is still no effective treatment available. Accordingly, there remains a need in the art for an effective treatment for MD.

C-Jun N-Terminal Kinase

Three c-Jun N-terminal kinase (JNK) enzymes have been identified. These represent alternatively spliced forms of three different genes: JNK1, JNK2, and JNK3 (Hibi M., Lin A., Smeal T., Minden A., Karin M. Genes Dev. 7:2135-2148, 1993; Mohit A. A., Martin M. H., and Miller C. A. Neuron 14:67-78, 1995; Gupta, S., Barrett, T., Whitmarsh, A. J., Cavanagh, J., Sluss, H. K., Derijard, B. and Davis, R. J. The EMBO J. 15:2760-2770, 1996). Activation of the JNK pathway has been documented in a number of disease settings, providing the rationale for targeting this pathway for drug discovery. In addition, molecular genetic approaches have validated the pathogenic role of the JNK pathway in several diseases. Many genes are regulated by the JNK pathway through activation of the transcription factors AP-1 and ATF-2, including TNF-alpha, IL-2, E-selectin, and matrix metalloproteinases such as collagenase-1 (Manning A. M. and Mercurio F., Exp Opin Invest Drugs, 6: 555-567, 1997).

### SUMMARY OF THE INVENTION

This invention encompasses methods for treating and/or preventing MD, which comprise administering to a patient in need thereof an effective amount of a JNK Inhibitor. The invention also encompasses methods for managing MD (e.g., lengthening the time of remission), which comprise administering to a patient in need of such management an effective amount of a JNK Inhibitor.

Another embodiment of the invention encompasses the use of an effective amount of a JNK Inhibitor in combination with another therapeutic agent useful to treat, prevent and/or manage MD such as, but not limited to, a steroid, a light sensitizer, an integrin, an antioxidant, an interferon, a xanthine derivative, a growth hormone, a neutrotrophic factor, a regulator of neovascularization, an anti-VEGF antibody, a prostaglandin, an antibiotic, a phytoestrogen, an anti-inflammatory compound, an IMiD@, a SelCID@, or an antiangiogenesis compound, or a combination thereof.

Yet another embodiment of the invention encompasses methods for treating, preventing and/or managing MD, comprising administering to a patient in need thereof an effective amount of a JNK Inhibitor in combination with a conventional therapy used to treat or prevent MD such as, but not limited to, surgical intervention (e.g., laser photocoagulation therapy and photodynamic therapy).

The invention further encompasses pharmaceutical compositions, single unit dosage forms, and kits suitable for use in treating, preventing and/or managing MD, which comprise an effective amount of a JNK Inhibitor.

The following Detailed Description and Examples illustrate non-limiting embodiments of the invention.

Definitions

As used herein, the term “macular degeneration” or “MD” encompasses all forms of macular degenerative diseases regardless of a patient's age, although some macular degenerative diseases are more common in certain age groups. These include, but are not limited to, Best's disease or vitelliform (most common in patients under about seven years of age); Stargardt's disease, juvenile macular dystrophy or ftindus flavimaculatus (most common in patients between about five and about 20 years of age); Behr's disease, Sorsby's disease, Doyne's disease or honeycomb dystrophy (most common in patients between about 30 and about 50 years of age); and age-related macular degeneration (most common in patients of about 60 years of age or older). In one embodiment, the cause of the macular degenerative disease is genetic. In another embodiment, the cause of the macular degenerative disease is physical trauma. In another embodiment, the cause of the macular degenerative disease is diabetes. In another embodiment, the cause of the macular degenerative disease is malnutrition. In another embodiment, the cause of the macular degenerative disease is infection.

As used herein, the term “patient” means an animal (e.g., cow, horse, sheep, pig, chicken, turkey, quail, cat, dog, mouse, rat, rabbit or guinea pig), preferably a mammal such as a non-primate and a primate (e.g., monkey or human), most preferably a human.

“Alkyl” means a saturated straight chain or branched non-cyclic hydrocarbon having from 1 to 10 carbon atoms. “Lower alkyl” means alkyl, as defined above, having from 1 to 4 carbon atoms. Representative saturated straight chain alkyls include -methyl, -ethyl, -n-propyl, -n-butyl, -n-pentyl, -n-hexyl, -n-heptyl, -n-octyl, -nnonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3dimethylhexyl, 2,4-dimethylhexyl, 2,5-dimethylhexyl, 2,2-dimethylpentyl, 2,2dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like.

An “alkenyl group” or “alkylidene” mean a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-i -butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. An alkenyl group can be unsubstituted or substituted. A “cyclic alkylidene” is a ring having from 3 to 8 carbon atoms and including at least one carbon-carbon double bond, wherein the ring can have from 1 to 3 heteroatoms.

An “alkynyl group” means a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. An alkynyl group can be unsubstituted or substituted.

The terms “Halogen” and “Halo” mean fluorine, chlorine, bromine or iodine.

“Haloalkyl” means an alkyl group, wherein alkyl is defined above, substituted with one or more halogen atoms.

“Keto” means a carbonyl group (i.e., C═O).

“Acyl” means an —C(O)alkyl group, wherein alkyl is defined above, including —C(O)CH₃, —C(O)CH₂CH₃, —C(O)(CH₂)₂CH₃, —C(O)(CH₂)₃CH₃, —C(O)(CH₂)₄CH₃, —C(O)(CH₂)₅CH₃, and the like.

“Acyloxy” means an —OC(O)alkyl group, wherein alkyl is defined above, including —OC(O)CH₃, —OC(O)CH₂CH₃, —OC(O)(CH₂)₂CH₃, —OC(O)(CH₂)₃CH₃, —OC(O)(CH₂)₄CH₃, —OC(O)(CH₂)₅CH₃, and the like.

“Ester” means and —C(O)Oalkyl group, wherein alkyl is defined above, including —C(O)OCH₃, —C(O)OCH₂CH₃, —C(O)O(CH₂)₂CH₃, —C(O)O(CH₂)₃CH₃, —C(O)O(CH₂)₄CH₃, —C(O)O(CH₂)₅CH₃, and the like.

“Alkoxy” means —O-(alkyl), wherein alkyl is defined above, including —OCH₃, —OCH₂CH₃, —O(CH₂)₂CH₃, —O(CH₂)₃CH₃, —O(CH₂)₄CH₃, —O(CH₂)₅CH₃, and the like. “Lower alkoxy” means —O—(lower alkyl), wherein lower alkyl is as described above.

“Alkoxyalkoxy” means —O-(alkyl)-O-(alkyl), wherein each alkyl is independently an alkyl group defined above, including —OCH₂OCH₃, —OCH₂CH₂OCH₃, —OCH₂CH₂OCH₂CH₃, and the like.

“Alkoxycarbonyl” means —C(═O)O-(alkyl), wherein alkyl is defined above, including —C(═O)O—CH₃, —C(═O)O—CH₂CH₃, —C(═O)O—(CH₂)₂CH₃, —C(═O)O(CH₂)₃CH₃, —C(═O)O—(CH₂)₄CH₃, —C(═O)O—(CH₂)₅CH₃, and the like.

“Alkoxycarbonylalkyl” means -(alkyl)-C(═O)O-(alkyl), wherein each alkyl is independently defined above, including —CH₂—C(═O)O—CH₃, —CH₂—C(═O)OCH₂CH₃, —CH₂—C(═O)O—(CH₂)₂CH₃, —CH₂—C(═O)O—(CH₂)₃CH₃, —CH₂—C(═O)O(CH₂)₄CH₃, —CH₂—C(═O)O—(CH₂)₅CH₃, and the like.

“Alkoxyalkyl” means -(alkyl)-O-(alkyl), wherein each alkyl is independently an alkyl group defined above, including —CH₂OCH₃, —CH₂OCH₂CH₃, —(CH₂)₂OCH₂CH₃, —(CH₂)₂O(CH₂)₂CH₃, and the like.

“Aryl” means a carbocyclic aromatic group containing from 5 to 10 ring atoms. Representative examples include, but are not limited to, phenyl, tolyl, anthracenyl, fluorenyl, indenyl, azulenyl, pyridinyl and naphthyl, as well as benzo-fused carbocyclic moieties including 5,6,7,8-tetrahydronaphthyl. A carbocyclic aromatic group can be unsubstituted or substituted. In one embodiment, the carbocyclic aromatic group is a phenyl group.

“Aryloxy” means —O-aryl group, wherein aryl is as defined above. An aryloxy group can be unsubstituted or substituted. In one embodiment, the aryl ring of an aryloxy group is a phenyl group

“Arylalkyl” means -(alkyl)-(aryl), wherein alkyl and aryl are as defined above, including —(CH₂)phenyl, —(CH₂)₂phenyl, —(CH₂)₃phenyl, —CH(phenyl)₂, —CH(phenyl)₃, —(CH₂)tolyl, —(CH₂)anthracenyl, —(CH₂)fluorenyl, —(CH₂)indenyl, —(CH₂)azulenyl, —(CH₂)pyridinyl, —(CH₂)naphthyl, and the like.

“Arylalkyloxy” means —O-(alkyl)-(aryl), wherein alkyl and aryl are defined above, including —O—(CH₂)₂phenyl, —O—(CH₂)₃phenyl, —O—CH(phenyl)₂, —O—CH(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)pyridinyl, —O—(CH₂)naphthyl, and the like.

“Aryloxyalkyl” means -(alkyl)-O—(aryl), wherein alkyl and aryl are defined above, including —CH₂—O-(phenyl), —(CH₂)₂—O-phenyl, —(CH₂)₃—O-phenyl, —(CH₂)—O-tolyl, —(CH₂)—O-anthracenyl, —(CH₂)—O-fluorenyl, —(CH₂)—O-indenyl, —(CH₂)—O—azulenyl, —(CH₂)—O-pyridinyl, —(CH₂)—O-naphthyl, and the like.

“Cycloalkyl” means a monocyclic or polycyclic saturated ring having carbon and hydrogen atoms and having no carbon-carbon multiple bonds. Examples of cycloalkyl groups include, but are not limited to, (C₃-C₇)cycloalkyl groups, including cyclopropyl, cyclobutyl, cyclopentyl, cyclohexyl, and cycloheptyl, and saturated cyclic and bicyclic terpenes. A cycloalkyl group can be unsubstituted or substituted. In one embodiment, the cycloalkyl group is a monocyclic ring or bicyclic ring.

“Cycloalkyloxy” means —O—(cycloalkyl), wherein cycloalkyl is defined above, including —O-cyclopropyl, —O-cyclobutyl, —O-cyclopentyl, —O-cyclohexyl, —O-cycloheptyl and the like.

“Cycloalkylalkyloxy” means —O-(alkyl)-(cycloalkyl), wherein cycloalkyl and alkyl are defined above, including —O—CH₂-cyclopropyl, —O—(CH₂)₂-cyclopropyl, —O(CH₂)₃-cyclopropyl, —O—(CH₂)₄-cyclopropyl, O—CH₂-cyclobutyl, O—CH₂-cyclopentyl, —O—CH₂-cyclohexyl, (CH₂-cycloheptyl, and the like.

“Aminoalkoxy” means —O-(alkyl)-NH₂, wherein alkyl is defined above, such as —O—CH₂—NH₂, —O—(CH₂)₂—NH₂, —O—(CH₂)₃—NH₂, —O—(CH₂)₄—NH₂, —O—(CH₂)₅—NH₂, and the like.

“Mono-alkylarnino” means —NH(alkyl), wherein alkyl is defined above, such as —NHCH₃, —NHCH₂CH₃, —NH(CH₂)₂CH₃, —NH(CH₂)₃CH₃, —NH(CH₂)₄CH₃, —NH(CH₂)₅CH₃, and the like.

“Di-alkylamino” means -N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃), and the like.

“Mono-alkylaminoalkoxy” means —O-(alkyl)-NH(alkyl), wherein each alkyl is independently an alkyl group defined above, including —O—(CH₂)—NHCH₃, —O—(CH₂)—NHCH₂CH₃, —O—(CH₂)—NH(CH₂)₂CH₃, —O—(CH₂)—NH(CH₂)₃CH₃, —O—(CH₂)

NH(CH₂)₄CH₃, —O—(CH₂)—NH(CH₂)₅CH₃, —O—(CH₂)₂—NHCH₃, and the like.

“Di-alkylaminoalkoxy” means —O-(alkyl)-N(alkyl)(alkyl), wherein each alkyl is independently an alkyl group defined above, including —O—(CH₂)—N(CH₃)₂, —O(CH₂)—N(CH₂CH₃)₂, —O—(CH₂)—N((CH₂)₂CH₃)₂, —O—(CH₂)—N(CH₃)(CH₂CH₃), and the like.

“Arylamino” means —NH(aryl), wherein aryl is defined above, including —NH(phenyl), —NH(tolyl), —NH(anthracenyl), —NH(fluorenyl), —NH(indenyl), —NH(azulenyl), —NH(pyridinyl), —NH(naphthyl), and the like.

“Arylalkylamino” means —NH-(alkyl)-(aryl), wherein alkyl and aryl are defined above, including —NH—CH₂-(phenyl), —NH—CH₂-(tolyl), —NH—CH₂-(anthracenyl), —NH—CH₂-(fluorenyl), —NH—CH₂-(indenyl), —NH—CH₂-(azulenyl), —NH—CH₂-(Pyridinyl), —NH—CH₂-(naphthyl), —NH-(CH₂)₂-(phenyl) and the like.

“Alkylamino” means mono-alkylamino or di-alkylamino as defined above, such as —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and —N(alkyl)(alkyl), wherein each alkyl is independently an alkyl group defined above, including —N(CH₃)₂, —N(CH₂CH₃)₂, —N((CH₂)₂CH₃)₂, —N(CH₃)(CH₂CH₃) and the like.

“Cycloalkylamino” means —NH-(cycloalkyl), wherein cycloalkyl is as defined above, including —NH-cyclopropyl, —NH-cyclobutyl, —NH-cyclopentyl, —NH—cyclohexyl, —NH-cycloheptyl, and the like.

“Carboxyl” and “carboxy” mean —COOH.

“Cycloalkylalkylamino” means —NH-(alkyl)-(cycloalkyl), wherein alkyl and cycloalkyl are defined above, including —NH—CH₂-cyclopropyl, —NH—CH₂cyclobutyl, —NH—CH₂-cyclopentyl, —NH—CH₂-cyclohexyl, —NH—CH₂-cycloheptyl, —NH—(CH₂)₂-cyclopropyl and the like.

“Aminoalkyl” means -(alkyl)-NH₂, wherein alkyl is defined above, including CH₂—NH₂, —(CH₂)₂—NH₂, —(CH₂)₃—NH₂, —(CH₂)₄—NH₂, —(CH₂)₅—NH₂ and the like.

“Mono-alkylaminoalkyl” means -(alkyl)-NH(alkyl),wherein each alkyl is independently an alkyl group defined above, including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄CH₃, —CH₂NH(CH₂)₅CH₃, —(CH₂)₂—NH—CH₃, and the like.

“Di-alkylaminoalkyl” means -(alkyl)-N(alkyl)(alkyl),wherein each alkyl is independently an alkyl group defined above, including —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, oxetanyl, azepinyl, piperazinyl, morpholinyl, dioxanyl, thietanyl and oxazolyl.

“Heteroarylalkyl” means -(alkyl)-(heteroaryl), wherein alkyl and heteroaryl are defined above, including —CH₂-triazolyl, —CH₂-tetrazolyl, —CH₂-oxadiazolyl, —CH₂-pyridyl, —CH₂-furyl, —CH₂-benzofuranyl, —CH₂-thiophenyl, —CH₂-benzothiophenyl, —CH₂-quinolinyl, —CH₂-pyrrolyl, —CH₂-indolyl, —CH₂-oxazolyl, —CH₂-benzoxazolyl, —CH₂-imidazolyl, —CH₂-benzimidazolyl, —CH₂-thiazolyl, —CH₂-benzothiazolyl, —CH₂-isoxazolyl, —CH₂-pyrazolyl, —CH₂-isothiazolyl, —CH₂-pyridazinyl, —CH₂-pyrimidinyl, —CH₂-pyrazinyl, —CH₂-triazinyl, —CH₂-cinnolinyl, —CH₂-phthalazinyl, —CH₂-quinazolinyl, —CH₂-pyrimidyl, —CH₂-oxetanyl, —CH₂-azepinyl, —CH₂-piperazinyl, —CH₂-morpholinyl, —CH₂-dioxanyl, —CH₂-thietanyl, —CH₂-oxazolyl, —(CH₂)₂-triazolyl, and the like.

“Heterocycle” means a 5- to 7-membered monocyclic, or 7- to 10-membered bicyclic, heterocyclic ring which is either saturated, unsaturated, and which contains from 1 to 4 heteroatoms independently selected from nitrogen, oxygen and sulfur, and wherein the nitrogen and sulfur heteroatoms can be optionally oxidized, and the nitrogen heteroatom can be optionally quatemized, including bicyclic rings in which any of the above heterocycles are fused to a benzene ring. The heterocycle can be attached via any heteroatom or carbon atom. Heterocycles include heteroaryls as defined above. Representative heterocycles include morpholinyl, pyrrolidinonyl, pyrrolidinyl, piperidinyl, hydantoinyl, valerolactamyl, oxinanyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocycle fused to phenyl” means a heterocycle, wherein heterocycle is defined as above, that is attached to a phenyl ring at two adjacent carbon atoms of the phenyl ring.

“Heterocycloalkyl” means -(alkyl)-(heterocycle), wherein alkyl and heterocycle are defined above, including —CH₂-morpholinyl, —CH₂-pyrrolidinonyl, —CH₂-pyrrolidinyl, —CH₂-piperidinyl, —CH₂-hydantoinyl, —CH₂-valerolactamyl, —CH₂-oxinanyl, —CH₂-oxetanyl, —CH₂-tetrahydrofuranyl, —CH₂-tetrahydropyranyl, —CH₂-tetrahydropyridinyl, —CH₂-tetrahydroprimidinyl, —CH₂-tetrahydrothiophenyl, —CH₂-tetrahydrothiopyranyl, —CH₂-tetrahydropyrimidinyl, —CH₂-tetrahydrothiophenyl, —CH₂-tetrahydrothiopyranyl, and the like.

The term “substituted” as used herein means any of the above groups (i.e., aryl, arylalkyl, heterocycle and heterocycloalkyl) wherein at least one hydrogen atom of the moiety being substituted is replaced with a substituent. In one embodiment, each carbon atom of the group being substituted is substituted with no more that two substituents. In another embodiment, each carbon atom of the group being substituted is substituted with no more than one substituent. In the case of a keto substituent, two hydrogen atoms are replaced with an oxygen which is attached to the carbon via a double bond. Substituents include halogen, hydroxyl, alkyl, haloalkyl, mono- or di-substituted aminoalkyl, alkyloxyalkyl, aryl, arylalkyl, heterocycle, heterocycloalkyl, —NRₐR_{b}, —NRₐC(═O)R_{b}, —NRₐC(═O)NRₐR_{b}, —NRₐC(═O)OR_{b}—NRₐSO₂R_{b}, —ORₐ, —C(═O)R₂ C(═O)ORₐ —C(═O)NRₐR_{b}, —OC(═O)Rₐ, —OC(═O)ORₐ, —OC(═O)NRₐR_{b}, —NRₐSO₂R_{b}, or a radical of the formula -Y—Z—Rₐ where Y is alkanediyl, or a direct bond, Z is —O—, —S—, —N(R_{b})—, —C(═O)—, —C(═O)O—, —OC(═O)—, —N(R_{b})C(═O)—, —C(═O)N(R_{b})— or a direct bond, wherein Rₐ and R_{b} are the same or different and independently hydrogen, amino, alkyl, haloalkyl, aryl, arylalkyl, heterocycle, or heterocylealkyl, or wherein Rₐ and R_{b} taken together with the nitrogen atom to which they are attached form a heterocycle.

“Haloalkyl” means alkyl, wherein alkyl is defined as above, having one or more hydrogen atoms replaced with halogen, wherein halogen is as defined above, including —CF₃, —CHF₂, —CH₂F, —CBr₃, —CHBr₂, —CH₂Br, —CC1₃, —CHC₁₂, —CH₂Cl, —C₁₃, —CH₁₂, —CH₂I, —CH₂—CF₃, —CH₂—CHF₂, —CH₂—CH₂F, —CH₂-CBr₃, —CH₂—CHBr₂, —CH₂—CH₂Br, —CH₂—CC₁₃, —CH₂—CHC₁₂, —CH₂—CH₂Cl, —CH₂-C₁₃, —CH₂—CHI₂, —CH₂—CH₂I, and the like.

“Hydroxyalkyl” means alkyl, wherein alkyl is as defined above, having one or more hydrogen atoms replaced with hydroxy, including —CH₂OH, —CH₂CH₂OH, —(CH₂)₂CH₂OH, —(CH₂)₃CH₂OH, —(CH₂)₄CH₂OH, —(CH₂)₅CH₂OH, —CH(OH)—CH₃, —CH₂CH(OH)CH₃, and the like.

“Hydroxy” means —OH.

“Sulfonyl” means —SO₃H.

“Sulfonylalkyl” means —SO₂-(alkyl), wherein alkyl is defined above, including —SO₂—CH₃, —SO₂—CH₂CH₃, —SO₂—(CH₂)₂CH₃, —SO₂—(CH₂)₃CH₃, —SO₂(CH₂)₄CH₃, —SO₂—(CH₂)₅CH₃, and the like.

“Sulfinylalkyl” means —SO-(alkyl), wherein alkyl is defined above, including —SO—CH₃, —SO—CH₂CH₃, —SO—(CH₂)₂CH₃, —SO—(CH₂)₃CH₃, —SO—(CH₂)₄CH₃, —SO—(CH₂)₅CH₃, and the like.

“Sulfonamidoalkyl” means —NHSO₂-(alkyl), wherein aklyl is defined above, including —NHSO₂—CH₃, —NHSO₂—CH₂CH₃, —NHSO₂—(CH₂)₂CH₃, —NHSO₂—(CH₂)₃CH₃, —NHSO₂—(CH₂)₄CH₃, —NHSO₂—(CH₂)₅CH₃, and the like.

“Thioalkyl” means -S-(alkyl), wherein alkyl is defined above, including —S—CH₃, —S—CH₂CH₃, —S—(CH₂)₂CH₃, —S—(CH₂)₃CH₃, —S—(CH₂)₄CH₃, —S—(CH₂)₅CH₃, and the like.

As used herein, the term “JNK Inhibitor” encompasses, but is not limited to, compounds disclosed herein. Without being limited by theory, specific JNK Inhibitors are capable of inhibiting the activity of JNK in vitro or in vivo. The JNK Inhibitor can be in the form of a pharmaceutically acceptable salt, free base, solvate, hydrate, stereoisomer, clathrate or prodrug thereof. Such inhibitory activity can be determined by an assay or animal model well-known in the art including those set forth in Section 5. In one embodiment, the JNK Inhibitor is a compound of structure (I)-(III).

As used herein, unless otherwise specified, the terms “prevent”, “preventing” or “prevention” include, but are not limited to, inhibiting MD or a symptom of MD. The symptoms of with MD include, but are not limited to, blindness, loss of central vision, blurred vision, wavy vision and blind spots.

As used herein, unless otherwise specified, the terms “treat”, “treating” or “treatment” refer to the eradication of MD or a symptom of MD. In one embodiment, “treat”, “treating” or “treatment” refer to minimizing the spread or minimizing the worsening of MD or a symptom of MD.

As used herein, the term “manage”, “managing” or “management” when used in connection with MD refer to providing beneficial effects to a patient being administered a JNK Inhibitor, which does not result in a cure of MD. In certain embodiments, a patient is administered one or more JNK Inhibitors to manage MD so as to prevent the progression or worsening of MD.

“JNK” means a protein or an isoform thereof expressed by a JNK 1, JNK 2, or JNK 3 gene (Gupta, S., Barrett, T., Whitmarsh, A. J., Cavanagh, J., Sluss, H. K., Derijard, B. and Davis, R. J. The EMBO J. 15:2760-2770 (1996)).

As used herein, the phrase “an effective amount” when used in connection with a JNK Inhibitor means an amount of the JNK Inhibitor that is usefuil for treating or preventing MD.

As used herein, the phrase “an effective amount” when used in connection with a second active agent means an amount of the second active agent that is usefuil for for treating or preventing MD.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the JNK Inhibitor include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995).

As used herein and unless otherwise indicated, the term “polymorph” means a particular crystalline arrangement of the JNK Inhibitor. Polymorphs can be obtained through the use of different work-up conditions and/or solvents. In particular, polymorphs can be prepared by recrystallization of a JNK Inhibitor in a particular solvent.

As used herein and unless otherwise indicated, the term “prodrug” means a JNK Inhibitor derivative that can hydrolyze, oxidize, or otherwise react under biological conditions (in vitro or in vivo) to provide an active compound, particularly a JNK Inhibitor. Examples of prodrugs include, but are not limited to, derivatives and metabolites of a JNK Inhibitor that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure a compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

### DETAILED DESCRIPTION OF THE INVENTION

Illustrative JNK Inhibitors

As mentioned above, the present invention is directed to methods useful for treating, preventing and/or managing MD, comprising administering an effective amount of a JNK Inhibitor to a patient in need thereof. Illustrative JNK Inhibitors are set forth below.

In one embodiment, the JNK Inhibitor has the following structure (I):

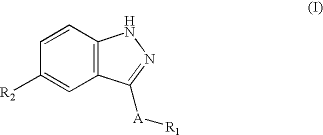

wherein:

A is a direct bond, —(CH₂)ₐ—, —(CH₂)_{b}CH═CH(CH₂)_{c}—, or —(CH₂)_{b}C|C(CH₂)_{c}—;

R₁ is aryl, heteroaryl or heterocycle fused to phenyl, each being optionally substituted with one to four substituents independently selected from R₃;

R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)CC(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R_{6;}

a is 1, 2, 3, 4, 5 or 6;

b and c are the same or different and at each occurrence independently selected from 0, 1, 2, 3 or 4;

d is at each occurrence 0, 1 or 2;

R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, C(═O)NR₈OR₉, —SO₂NR₈R₉, —NR₈SO₂R₉, —CN, —NO₂, —NR₈R₉, —NR₈C(═O)R₉, NR₈C(═O)(CH₂)_{b}OR₉, —NR₈C(═O)(CH₂)_{b}R₉, —O(CH₂)_{b}NR₈R₉, or heterocycle fused to phenyl;

R₄ is alkyl, aryl, arylalkyl, heterocycle or heterocycloalkyl, each being optionally substituted with one to four substituents independently selected from R₃, or R₄ is halogen or hydroxy;

R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and

R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

In one embodiment, —A—R₁ is phenyl, optionally substituted with one to four substituents independently selected from halogen, alkoxy, —NR₈C(═O)R₉, —C(═O)NR₈R₉, and —O(CH₂)_{b}NR₉R₉, wherein b is 2 or 3 and wherein R₈ and R₉ are defined above.

In another embodiment, R₂ is —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆, and b is an integer ranging from 0-4.

In another embodiment, R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment, R₂ is 3-triazolyl or 5-tetrazolyl.

In another embodiment:

(a) —A—R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In another embodiment:

(a) —A—R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is 3-triazolyl or 5-tetrazolyl.

In another embodiment, R₂ is R₄, and R₄ is 3-triazolyl, optionally substituted at its 5-position with:

(a) a C—C₄ straight or branched chain alkyl group optionally substituted with a hydroxyl, methylamino, dimethylamino or 1-pyrrolidinyl group; or

(b) a 2-pyrrolidinyl group.

In another embodiment, R₂ is R₄, and R₄ is 3-triazolyl, optionally substituted at its 5-position with: methyl, n-propyl, isopropyl, 1-hydroxyethyl, 3-hydroxypropyl, methylaminomethyl, dimethylaminomethyl, 1-(dimethylamino)ethyl, 1-pyrrolidinylmethyl or 2-pyrrolidinyl.

In another embodiment, the compounds of structure (I) have structure (IA) when A is a direct bond, or have structure (IB) when A is —(CH₂)ₐ—:

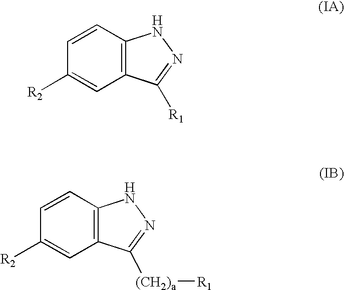

In other embodiments, the compounds of structure (I) have structure (IC) when A is a —CH₂)_{b}CH═CH(CH₂)_{c}—, and have structure (ID) when A is —(CH₂)_{b}C C(CH₂)_{c}—:

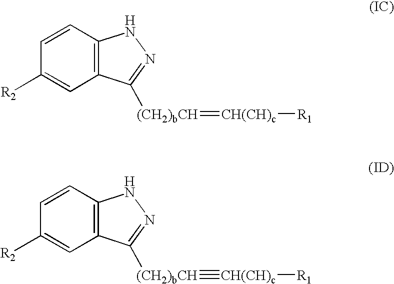

In further embodiments of this invention, R₁ of structure (I) is aryl or substituted aryl, such as phenyl or substituted phenyl as represented by the following structure (IE):

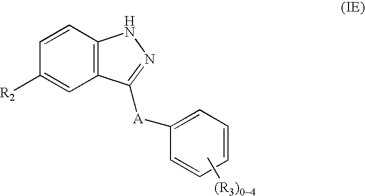

In another embodiment, R₂ of structure (I) is —(CH₂)_{b}NR₄(C═O)R₅. In one aspect of this embodiment, b=0 and the compounds have the following structure (IF):

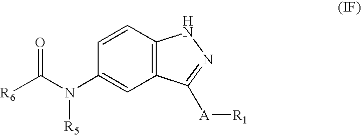

Representative R₂ groups of the compounds of structure (I) include alkyl (such as methyl and ethyl), halo (such as chloro and fluoro), haloalkyl (such as trifluoromethyl), hydroxy, alkoxy (such as methoxy and ethoxy), amino, arylalkyloxy (such as benzyloxy), mono- or di-alkylamine (such as —NHCH₃, —N(CH₃)₂ and —NHCH₂CH₃), —NHC(═O)R₄ wherein R₆ is a substituted or unsubstituted phenyl or heteroaryl (such as phenyl or heteroaryl substituted with hydroxy, carboxy, amino, ester, alkoxy, alkyl, aryl, haloalkyl, halo, —CONH₂ and —CONH alkyl), —NH(heteroarylalkyl) (such as —NHCH₂(3-pyridyl), —NHCH₂(4-pyridyl), heteroaryl (such as pyrazolo, triazolo and tetrazolo), —C(—O)NHR₆ wherein R₆ is hydrogen, alkyl, or as defined above (such as C(═O)NH₂, —C(═O)NHCH₃, —C(═O)NH(H-carboxyphenyl), —C(═O)N(CH₃)₂), arylalkenyl (such as phenylvinyl, 3-nitrophenylvinyl, 4-carboxyphenylvinyl), heteroarylalkenyl (such as 2-pyridylvinyl, 4-pyridylvinyl).

Representative R₃ groups of the compounds of structure (I) include halogen (such as chloro and fluoro), alkyl (such as methyl, ethyl and isopropyl), haloalkyl (such as trifluoromethyl), hydroxy, alkoxy (such as methoxy, ethoxy, npropyloxy and isobutyloxy), amino, mono- or di-alkylamino (such as dimethylamine), aryl (such as phenyl), carboxy, nitro, cyano, sulfinylalkyl (such as methylsulfinyl), sulfonylalkyl (such as methylsulfonyl), sulfonamidoalkyl (such as —NHSO₂CH₃), —NR₈C(═O)(CH₂)_{b}OR₉ (such as NHC(═O)CH₂OCH₃), NHC(═O)R₉ (such as —NHC(═O)CH₃, —NHC(═O)CH₂C₆H₅, —NHC(═O)(2-furanyl)), and —O(CH₂)_{b}NR₈R₉ (such as —O(CH₂)₂N(CH₃)₂).

The compounds of structure (I) can be made using organic synthesis techniques known to those skilled in the art, as well as by the methods described in International Publication No. WO 02/10137 (particularly in Examples 1-430, at page 35, line 1 to page 396, line 12), published Feb. 7, 2002, which is incorporated herein by reference in its entirety. Further, specific examples of these compounds are found in this publication.

Illustrative examples of JNK Inhibitors of structure (I) are:

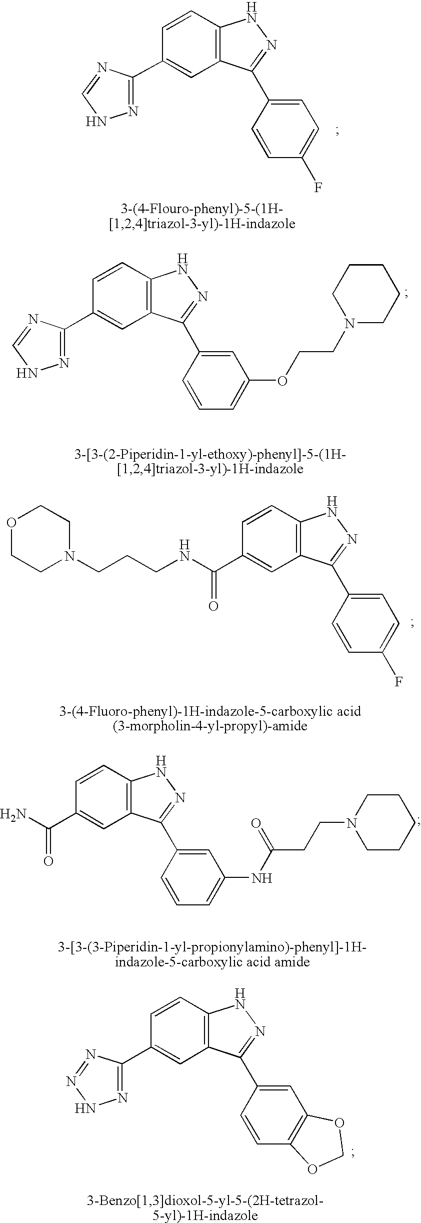

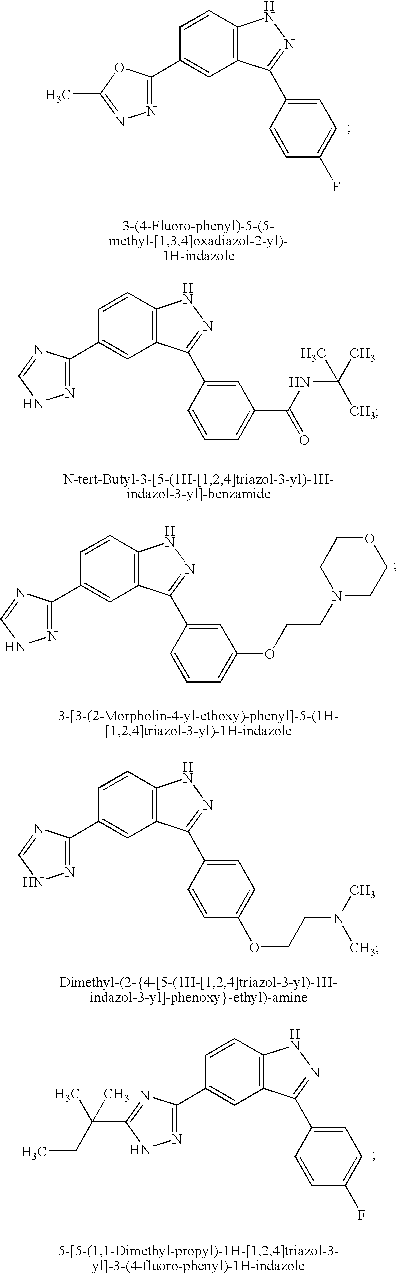

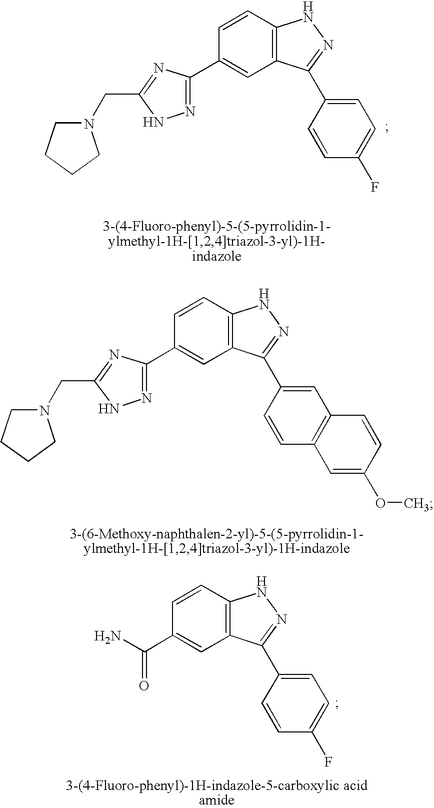

and pharmaceutically acceptable salts thereof.

In another embodiment, the JNK Inhibitor has the following structure (II):

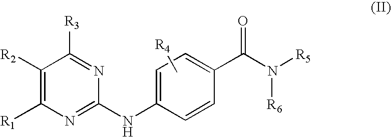

wherein:

R₁ is aryl or heteroaryl optionally substituted with one to four substituents independently selected from R₇;

R₂ is hydrogen;

R₃ is hydrogen or lower alkyl;

R₄ represents one to four optional substituents, wherein each substituent is the same or different and independently selected from halogen, hydroxy, lower alkyl and lower alkoxy;

R₅ and R₆ are the same or different and independently —R₈, —(CH₂)ₐC(═O)R₉, (CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNRIIC(═O)NR₉R₁₀, —(CH₂)ₐNR₉R₁₀, —(CH₂)OR₉, —(CH₂)ₐSO_{c}R₉ or —(CH₂)ₐSO₂NR₉R₁₀;

or R₅ and R₆ taken together with the nitrogen atom to which they are attached to form a heterocycle or substituted heterocycle;

R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, substituted heterocycle, heterocycloalkyl, _C(═O)OR₈, —OC(—O)R₈, —C(═O)NR₈R₉, —C(═O)NR₈OR₉, —SOCR₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, _O(CH₂)_{b}NR₉R₉, or heterocycle fused to phenyl;

R₈, R₉, R₁₀ and R₁₁ are the same or different and at each occurrence independently hydrogen, alkyl, aryl, arylalkyl, heterocycle, heterocycloalkyl;

or R₈ and R₉ taken together with the atom or atoms to which they are attached to form a heterocycle;

a and b are the same or different and at each occurrence independently selected from 0, 1, 2, 3 or 4; and

c is at each occurrence 0, 1 or 2.

In one embodiment, R₁ is a substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl or quinazolinyl.

In another embodiment R₁ is substituted or unsubstituted aryl or heteroaryl. When R₁ is substituted, it is substituted with one or more substituents defined below. In one embodiment, when substituted, R₁ is substituted with a halogen, —SO₂R₈ or —SO₂R₈R₉.

In another embodiment, R₁ is substituted or unsubstituted aryl, preferably phenyl. When R₁ is a substituted aryl, the substituents are defined below. In one embodiment, when substituted, R₁ is substituted with a halogen, —SO₂R₈ or —SO₂R₈R₉.

In another embodiment, R₅ and R₆, taken together with the nitrogen atom to which they are attached form a substituted or unsubstituted nitrogen-containing nonaromatic heterocycle, in one embodiment, piperazinyl, piperidinyl or morpholinyl.

When R₅ and R₆, taken together with the nitrogen atom to which they areattached form substituted piperazinyl, piperadinyl or morpholinyl, the piperazinyl, piperadinyl or morpholinyl is substituted with one or more substituents defined below. In one embodiment, when substituted, the substituent is alkyl, amino, alkylamino, alkoxyalkyl, acyl, pyrrolidinyl or piperidinyl.

In one embodiment, R₃ is hydrogen and R₄ is not present, and the JNK Inhibitor has the following structure (IIA):

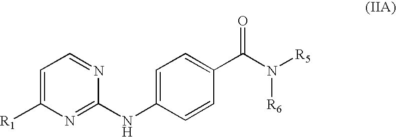

and pharmaceutically acceptable salts thereof.

In a more specific embodiment, R₁ is phenyl optionally substituted with R₇, and having the following structure (IIB):

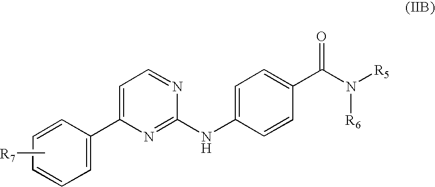

and pharmaceutically acceptable salts thereof.

In still a further embodiment, R₇ is at the para position of the phenyl group relative to the pyrimidine, as represented by the following structure (IIC):

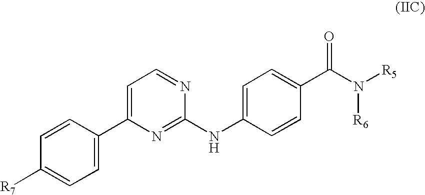

and pharmaceutically acceptable salts thereof.

The JNK Inhibitors of structure (II) can be made using organic synthesis techniques known to those skilled in the art, as well as by the methods described in International Publication No. WO 02/46170 (particularly Examples 1-27 at page 23, line 5 to page 183, line 25), published Jun. 13, 2002, which is hereby incorporated by reference in itsr entirety. Further, specific examples of these compounds are found in the publication.

Illustrative examples of JNK Inhibitors of structure (II) are:

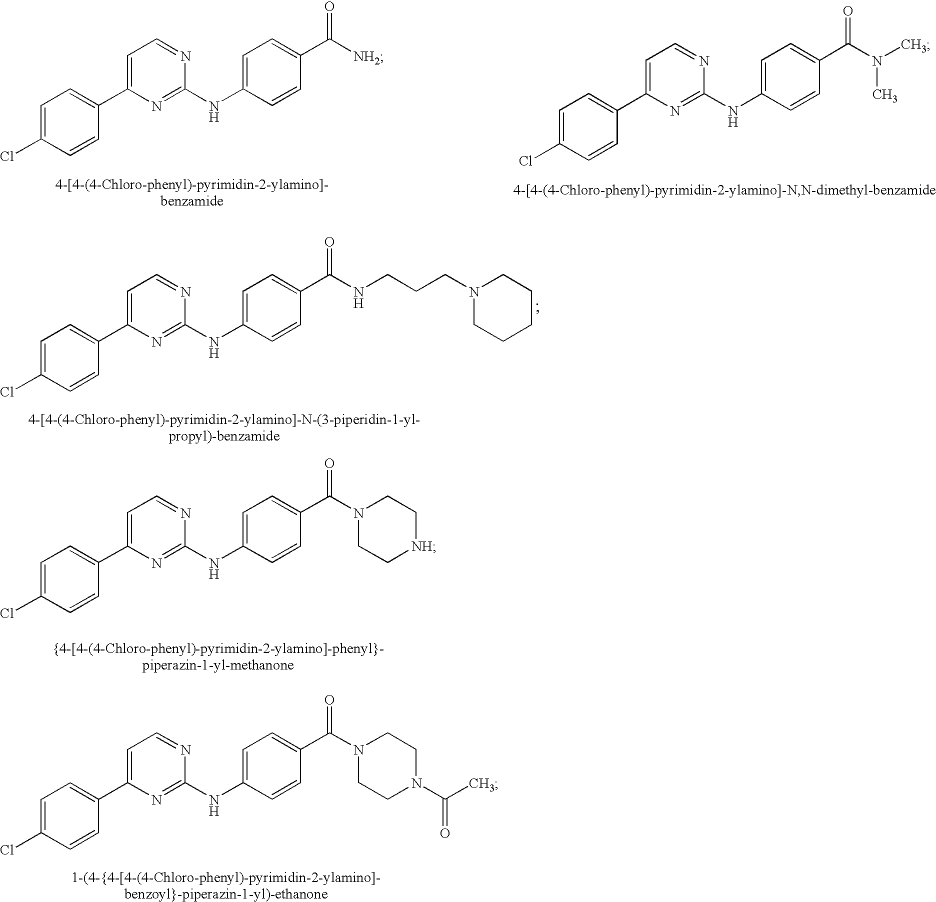

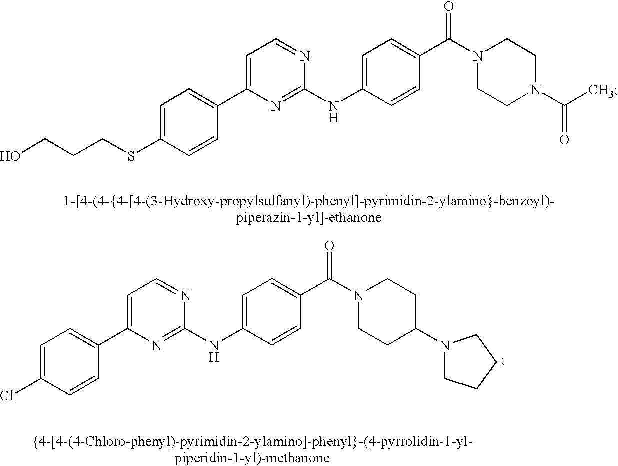

and pharmaceutically acceptable salts thereof.

In another embodiment, the JNK Inhibitor has the following structure (III):

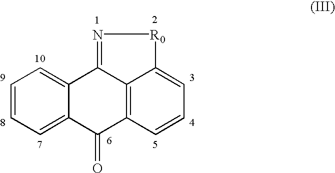

wherein R₀ is —O—, —S—, —S(O)—, —S(O)₂—, NH or —CH₂—;

the compound of structure (III) being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

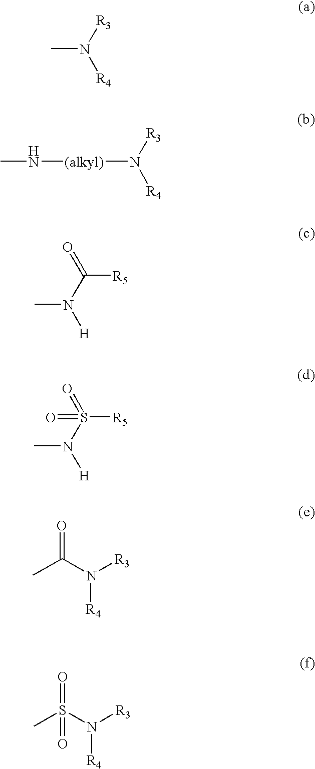

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

In another embodiment, the JNK Inhibitor has the following structure (IIIA):

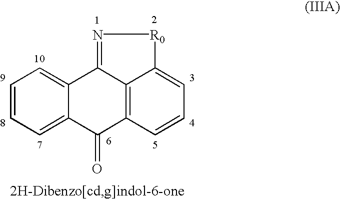

being: (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- alkylaminoalkoxy, di-alkylaminoalkoxy, or a group represented by structure (a), (b), (c), (d), (e), or (f):

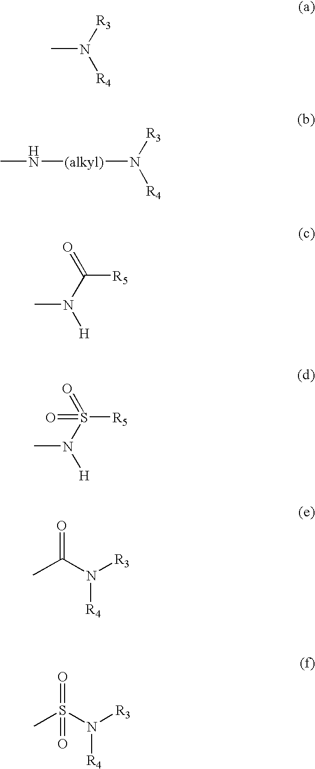

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

A subclass of the compounds of structure (IIIA) is that wherein the first or second substituent is present at the 5, 7, or 9 position. In one embodiment, the first or second substituent is present at the 5 or 7 position.

A second subclass of compounds of structure (IIIA) is that wherein the first or second substituent is present at the 5, 7, or 9 position;

the first or second substituent is independently alkoxy, aryloxy, aminoalkyl, mono-alkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK Inhibitor has the following structure (IIIB):

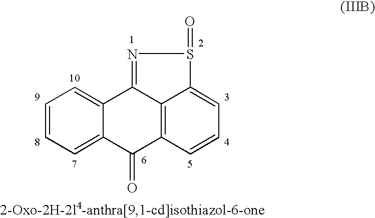

being (i) unsubstituted, (ii) monosubstituted and having a first substituent, or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b) (c), (d), (e), or (f):

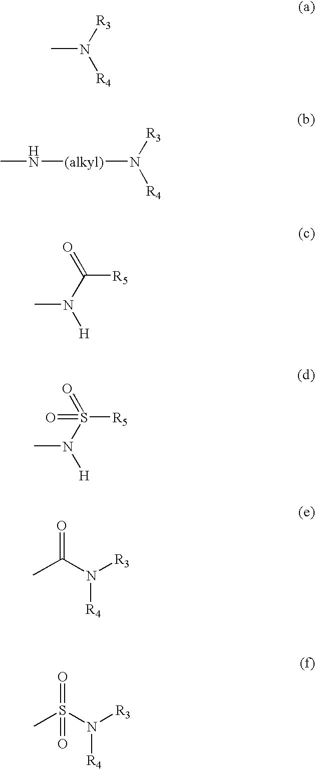

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylaamino, cycloalkylamino, cycloalkylalkylamino, aminoalkyl, mono-alkylaminoalkyl, or di-alkylaminoalkyl.

A subclass of the compounds of structure (IIIB) is that wherein the first or second substituent is present at the 5, 7, or 9 position. In one embodiment, the first or second substituent is present at the 5 or 7 position.

A second subclass of the compounds of structure (I113) is that wherein the first or second substituent is independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK Inhibitor has the following structure (IIIC):

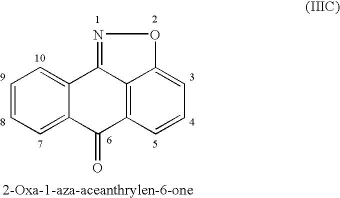

being (i) monosubstituted and having a first substituent or (ii) disubstituted and having a first substituent and a second substituent;

the first or second substituent, when present, is at the 3, 4, 5, 7, 8, 9, or 10 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c) (d), (e), or (f):

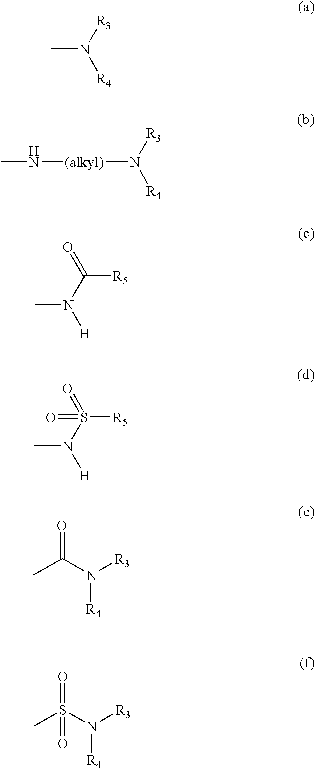

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylaamino, cycloalkylalkylamino, aminoalkyl, monoalkylaminoalkyl, or di-alkylaminoalkyl.

A subclass of the compounds of structure (IIIC) is that wherein the first or second substituent is present at the 5, 7, or 9 position. In one embodiment, the first or second substituent is present at the 5 or 7 position.

A second subclass of the compounds of structure (IIIC) is that wherein the first or second substituent is independently alkoxy, aryloxy, aminoalkyl, monoalkylaminoalkyl, di-alkylaminoalkyl, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, or cycloalkylalkyl.

In another embodiment, the JNK Inhibitor has the following structure (IIID):

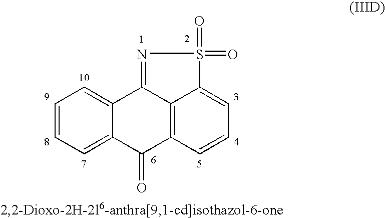

being (i) monosubstituted and having a first substituent present at the 5, 7, or 9 position, (ii) disubstituted and having a first substituent present at the 5 position and a second substituent present at the 7 position, (iii) disubstituted and having a first substituent present at the 5 position and a second substituent present at the 9 position, or (iv) disubstituted and having a first substituent present at the 7 position and a second substituent present at the 9 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

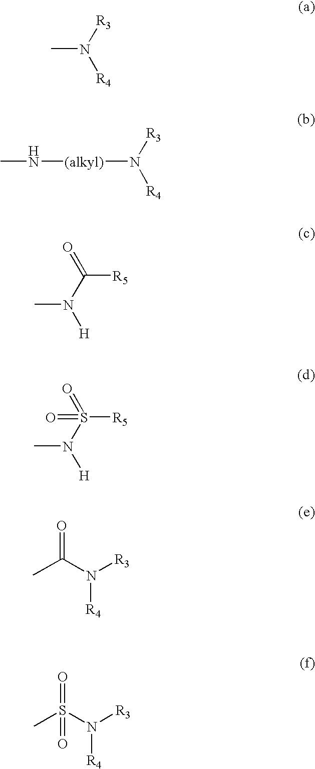

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

A subclass of the compounds of structure (IIID) is that wherein the first or second substituent is present at the 5 or 7 position.

A second subclass of the compounds of structure (IIID) is that wherein the first or second substituent is independently alkyl, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, dialkylaminoalkoxy, or a group represented by structure (a), (c), (d), (e), or (f).

Another subclass of the compounds of structure (IIID) is that wherein the first and second substituent are independently alkoxy, aryloxy, or a group represented by the structure (a), (c), (d), (e), or (f);

R₃ and R₄ are independently hydrogen, alkyl, cycloalkyl, aryl, arylalkyl, or cycloalkylalkyl; and

R₅ is hydrogen, alkyl, cycloalkyl, aryl, arylalkyl, alkoxycarbonyl, or cycloalkylalkyl.

In another embodiment, the JNK Inhibitor has the following structure (IIIE):

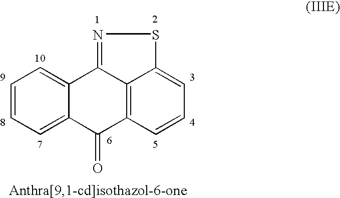

being (i) monosubstituted and having a first substituent present at the 5, 7, or 9 position, (ii) disubstituted and having a first substituent present at the 5 position and a second substituent present at the 9 position, (iii) disubstituted and having a first substituent present at the 7 position and a second substituent present at the 9 position, or (iv) disubstituted and having a first substituent present at the 5 position and a second substituent present at the 7 position;

wherein the first and second substituent, when present, are independently alkyl, halogen, hydroxy, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by structure (a), (b), (c), (d), (e), or (f):

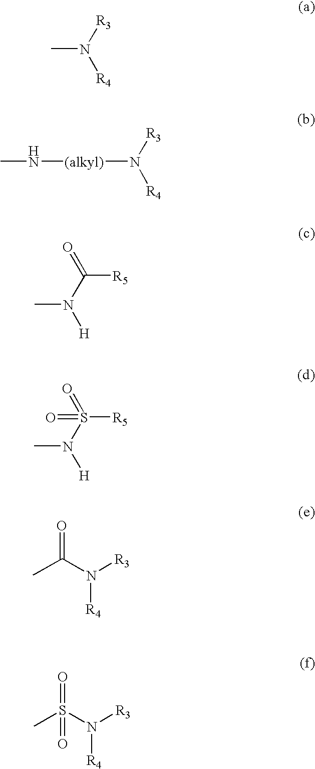

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, arninoalkyl, monoalkylaminoalkyl, or di-alkylaminoalkyl.

A subclass of the compounds of structure (IIIE) is that wherein the first or second substituent is present at the 5 or 7 position.

A second subclass of the compounds of structure (IIIE) is that wherein the compound of structure (IIIE) is disubstituted and at least one of the substituents is a group represented by the structure (d) or (f).

Another subclass of the compounds of structure (IIIE) is that wherein the compounds are monosubstituted. Yet another subclass of compounds is that wherein the compounds are monosubstituted at the 5 or 7 position with a group represented by the structure (e) or (f).

In another embodiment, the JNK Inhibitor has the following structure (IIIF):

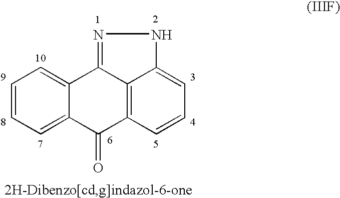

being (i) unsubstituted, (ii) monosubstituted and having a first substituent, or (iii) disubstituted and having a first substituent and a second substituent;

the first or second substituent, when present, is at the 3, 4, 5, 7, 8, 9, or 10 position;

wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono- alkylaminoalkoxy, dialkylaminoalkoxy, or a group represented by structure (a), (b), (c), (d), (e), or (f):

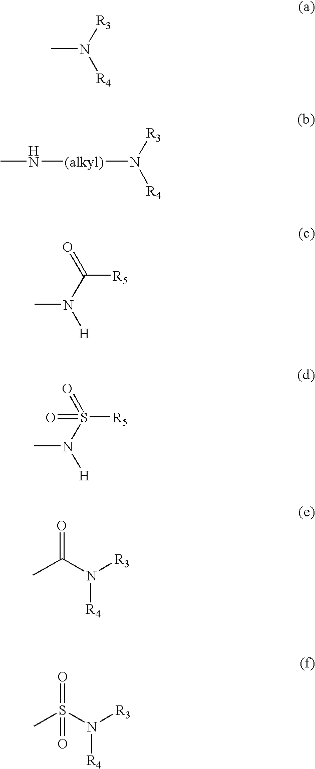

wherein R₃ and R₄ are taken together and represent alkylidene or a heteroatom-containing cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, mono-alkylaminoalkyl, or di-alkylaminoalkyl; and

R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylamino, cycloalkylamino, cycloalkylalkylamino, aminoalkyl, monoalkylaminoalkyl, or di-alkylaminoalkyl.

In one embodiment, the compound of structure (IIIF), or a pharmaceutically acceptable salt thereof is unsubstituted at the 3, 4, 5, 7, 8, 9, or 10 position.

The JNK Inhibitors of structure (III) can be made using organic synthesis techniques known to those skilled in the art, as well as by the methods described in International Publication No. WO 01/12609 (particularly Examples 1-7 at page 24, line 6 to page 49, line 16), published Feb. 22, 2001, as well as International Publication No. WO 02/066450 (particularly compounds AA-HG at pages 59-108), published Aug. 29, 2002, each of which is hereby incorporated by reference in its entirety. Further, specific examples of these compounds can be found in the publications.

Illustrative examples of JNK Inhibitors of structure (III) are:

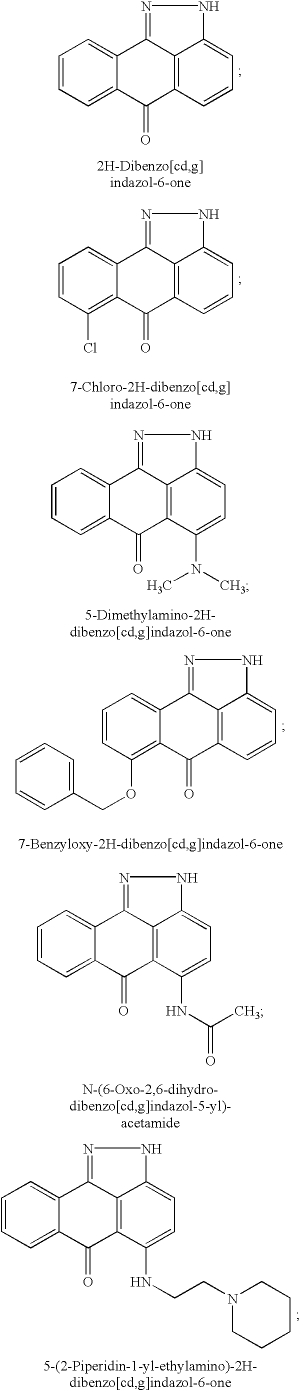

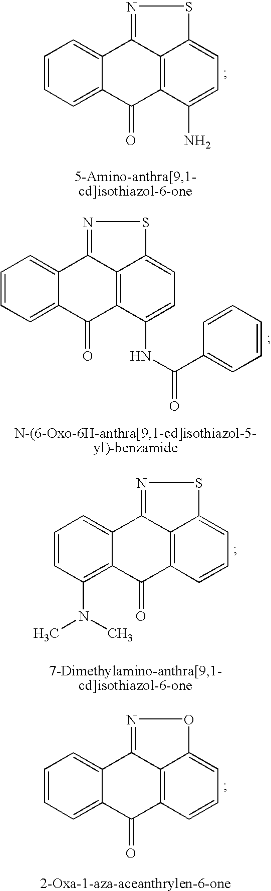

and pharmaceutically acceptable salts thereof.

Other JNK Inhibitors that are useful in the present methods include, but are not limited to, those disclosed in International Publication No. WO 00/39101, (particularly at page 2, line 10 to page 6, line 12); International Publication No. WO 01/14375 (particularly at page 2, line 4 to page 4, line 4); International Publication No. WO 00/56738 (particularly at page 3, line 25 to page 6, line 13); International Publication No. WO 01/27089 (particularly at page 3, line 7 to page 5, line 29); International Publication No. WO 00/12468 (particularly at page 2, line 10 to page 4, line 14); European Patent Publication 1 110 957 (particularly at page 19, line 52 to page 21, line 9); International Publication No. WO 00/75118 (particularly at page 8, line 10 to page 11, line 26); International Publication No. WO 01/12621 (particularly at page 8, line 10 to page 10, line 7); International Publication No. WO 00/64872 (particularly at page 9, line 1 to page, 106, line 2); International Publication No. WO 01/23378 (particularly at page 90, line 1 to page 91, linel 1); International Publication No. WO 02/16359 (particularly at page 163, line 1 to page 164, line 25); U.S. Pat. No. 6,288,089 (particularly at column 22, line 25 to column 25, line 35); U.S. Pat. No. 6,307,056 (particularly at column 63, line 29 to column 66, line 12); International Publication No. WO 00/35921 (particularly at page 23, line 5 to page 26, line 14); International Publication No. WO 01/91749 (particularly at page 29, lines 1-22); International Publication No. WO 01/56993 (particularly in at page 43 to page 45); and International Publication No. WO 01/58448 (particularly in at page 39), each of which is incorporated by reference herein in its entirety.

Pharmaceutical compositions including dosage forms of the invention, which comprise an effective amount of a JNK Inhibitor can be used in the methods of the invention.

Methods of Use

This invention encompass methods for treating, preventing and/or managing MD and related syndromes in a patient in need of such treatment, prevention and/or management comprising administration of an effective amount of a JNK Inhibitor.

The invention further encompasses methods for treating, preventing and/or managing MD and related syndromes in a patient with various stages and specific types of the disease, including, but not limited to, those referred to as wet MD, dry MD, age-related maculopathy (ARM), choroidal neovascularisation (CNVM), retinal pigment epithelium detachment (PED), and atrophy of retinal pigment epithelium (RPE). The invention further encompasses methods for treating a patient who has been previously treated for MD, is non-responsive to standard drug and non-drug-based MD treatments, as well as patient who has not previously been treated for MD. Because a patient with MD can have heterogenous clinical manifestations and varying clinical outcomes, the treatment given to a patient can vary, depending on his/her prognosis. The skilled clinician will be able to readily determine without undue experimentation specific secondary agents and treatments that can be effectively used to treat an individual patient.

In one embodiment, the duration of the administration of an effective amount of a JNK Inhibitor is about 2 to about 20 weeks. In another embodiment, the duration of the administration of an effective amount of a JNK Inhibitor is about 4 to about 16 weeks. In another embodiment, the duration of the administration of an effective amount of a JNK Inhibitor is about 8 to about 12 weeks. In another embodiment, an effective amount of the JNK Inhibitor is continued until the desired therapeutic effect is achieved.

In one embodiment, the MD is Best's disease or vitelliform (most common in patients under about 7 years of age).

In another embodiment, the MD is Stargardt's disease, juvenile macular dystrophy or fundus flavimaculatus (most common in patients between about 5 and about 20 years of age).

In another embodiment, the MD is Behr's disease, Sorsby's disease, Doyne's disease or honeycomb dystrophy (most common in patients between about 30 and about 50 years of age).

In another embodiment, the MD is age-related macular degeneration (most common in patients of about 60 years of age or older).

In one embodiment, the cause of the MD is genetic.

In another embodiment, the cause of the MD is physical trauma.

In another embodiment, the cause of the MD is diabetes.

In another embodiment, the cause of the MD is malnutrition.

In another embodiment, the cause of the MD is infection.

Combination Therapy with a Second Active Agent

The invention further encompasses methods for treating, preventing and/or managing MD and related syndromes in a patient in need of such treatment, prevention and/or management comprising the administration of an effective amount of a JNK Inhibitor and an effective amount of another active agent including, but not limited to, a steroid, a light sensitizer, an integrin, an antioxidant, an interferon, a xanthine derivative, a growth hormone, a neutrotrophic factor, a regulator of neovascularization, an anti-VEGF antibody, a prostaglandin, an antibiotic, a phytoestrogen, an antiinflammatory compound, IMiDs@ and SelCIDs@ (Celgene Corporation, New Jersey) (e.g., those disclosed in U.S. Pat. Nos. 6,075,041; 5,877,200; 5,698,579; 5,703,098; 6,429,221; 5,736,570; 5,658,940; 5,728,845; 5,728,844; 6,262,101; 6,020,358; 5,929,117; 6,326,388; 6,281,230; 5,635,517; 5,798,368; 6,395,754; 5,955,476; 6,403,613; 6,380,239; and 6,458,810, each of which is incorporated herein by reference), an antiangiogenesis compound or other conventional therapeutic agent known in the art to be useful for treating or preventing MD.

Examples of light sensitizers include, but are not limited to, verteporfin, tin etiopurpurin and motexafin lutetium.

Examples of xanthine derivatives include, but are not limited to, pentoxyfylline.

Examples of anti-VEGF antibodies include, but are not limited to, rhuFab.

Examples of steroids include, but are not limited to, 9-fluoro-11,21-dihydroxy-16,17-1-methylethylidinebis(oxy)pregna-1,4-diene-3,20-dione.

Examples of prostaglandins include, but are not limited to, prostaglandin F₂α derivatives such as latanoprost (see U.S. Pat. No. 6,225,348, which is incorporated by reference herein in its entirety).

Examples of antibiotics include, but are not limited to, tetracycline and its derivatives, rifamycin and its derivatives, macrolides, and metronidazole (see U.S. Pat. No. 6,218,369 and U.S. Pat. No. 6,015,803, which are incorporated by reference herein in their entirety).

Examples of phytoestrogens include, but are not limited to, genistein, genistin, 6′-O-Mal genistin, 6′-O-Ac genistin, daidzein, daidzin, 6′-O-Mal daidzin, 6′-O-Ac daidzin, glycitein, glycitin, 6′-O-Mal glycitin, biochanin A, formononetin and mixtures thereof (see U.S. Pat. No. 6,001,368, which is incorporated by reference herein in its entirety).

Examples of anti-inflammatory agents include, but are not limited to, triamcinolone acetomide and dexamethasone (see U.S. Pat. No. 5,770,589, which is incorporated by reference herein in its entirety).

Examples of antiangiogenesis compounds include, but are not limited to, thalidomide.

Examples of interferon include, but are not limited to, interferon-2α.

Examples of growth hormones include, but are not limited to, basic fibroblast growth factor (bFGF) and transforming growth factor β (TGF-β); neurotrophic factors, such as brain-derived neurotrophic factor (BDNF); and regulators of neovascularization, such as plasminogen activator factor type 2 (PAI-2).

Administration of a JNK Inhibitor and the other active agent can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. One route of administration for a JNK Inhibitor is oral. Routes of administration for the other active agent are known to those skilled in the art. See, e.g., Physicians' Desk Reference 1755-1760 (56^{th} ed. 2002).

In one embodiment of the invention, a JNK Inhibitor is administered by a parenteral, intravenous, subcutaneous, intradermal, intravitreal, topical, mucosal or oral route and in a single or divided effective daily dose in an amount of from about 0.1 mg to about 2500 mg, from about 1 mg to about 2000 mg, or from 10 mg to about 1500 mg, or from 50 mg to about 1000 mg, or from 100 mg to about 750 mg, or from 250 mg to about 500 mg.

In another embodiment, a JNK Inhibitor is administered in conjunction with the other active agent. The other active agent can be administered by a parenteral, intravenous, subcutaneous, intradermal, intravitreal, topical, mucosal or oral route and once or twice daily in an effective amount of from about 0.1 mg to about 2500 mg, from about 1 mg to about 2000 mg, or from 10 mg to about 1500 mg, or from 50 mg to about 1000 mg, or from 100 mg to about 750 mg, or from 250 mg to about 500 mg.

In further embodiments, the other active agent is administered weekly, monthly, bi-monthly or yearly. The specific amount of the other active agent can depend on the specific agent used, the type of MD being treated or prevented, the severity and stage of MD, and the amount(s) of a JNK Inhibitor and any optional other agent(s)ₐdministered to the patient.

In one embodiment, the JNK Inhibitor is administered to a patient as part of cycling therapy. Cycling therapy involves administration for a specified period of time, followed by administration for another specified period of time and repeating this sequential administration. Cycling therapy can reduce the development of resistance to one or more of the therapies, avoid or reduce the side effects of one of the therapies, and/or improve the efficacy of the treatment.

In one embodiment, a JNK Inhibitor is administered in a cycle of about 6 weeks, about once or twice every day. In another embodiment, a JNK Inhibitor is administered in a cycle of about 16 weeks, about once or twice every day. In another embodiment, a JNK Inhibitor is administered in a cycle of about 24 weeks, about once or twice every day. In another embodiment, a JNK Inhibitor is administered in a cycle of about 52 weeks, about once or twice every day. One administration cycle can comprise the administration of a JNK Inhibitor and at least one (1) or three (3) weeks of nonadministration. The number of cycles can range from about 1 to about 12 cycles, more typically from about 2 to about 10 cycles, and more typically from about 2 to about 8 cycles.

Combination Therapy With Other Therapies

In another embodiment, the invention encompasses methods for treating, preventing and/or managing MD, comprising administering to a patient in need thereof an effective amount of a JNK Inhibitor and an effective amount of light or laser therapy. Examples of light or laser therapy include, but are not limited to, laser photocoagulation therapy or photodynamic therapy. The JNK Inhibitor can be administered simultaneously or sequentially with the light or laser therapy. In one embodiment, the JNK Inhibitor is administered prior to light or laser therapy. In one embodiment, the JNK Inhibitor is administered about 4 weeks prior to light or laser therapy. In another embodiment, the JNK Inhibitor is administered about 2 weeks prior to light or laser therapy. In another embodiment, the JNK Inhibitor is administered about 1 weeks prior to light or laser therapy. In another embodiment, the JNK Inhibitor is administered just prior to or the day of light or laser therapy. In another embodiment, the JNK Inhibitor is administered after light or laser therapy. In another embodiment, the JNK Inhibitor is administered for about 1 week after light or laser therapy. In another embodiment, the JNK Inhibitor is administered for about 2 to about 8 weeks after light or laser therapy. In another embodiment, the JNK Inhibitor is administered for about 12 to about 16 weeks after light or laser therapy. In one embodiment, the JNK Inhibitor is administered during light or laser therapy.

In another embodiment, the invention encompasses methods for treating, preventing and/or managing MD, comprising administering to a patient in need thereof an effective amount of a JNK Inhibitor in combination with an ocular surgical procedure. The JNK Inhibitor can be administered simultaneously or sequentially with the ocular surgical procedure. In one embodiment, the JNK Inhibitor is administered prior to the ocular surgical procedure. In another embodiment, the JNK Inhibitor is administered after the ocular surgical procedure. In another embodiment, the JNK Inhibitor is administered during the ocular surgical procedure. In another embodiment, the JNK Inhibitor is administered before, during and after the ocular surgical procedure.

Pharmaceutical Compositions

The compositions comprising a JNK Inhibitor include bulk-drug compositions useful in the manufacture of pharmaceutical compositions (e.g., impure or non-sterile compositions) and pharmaceutical compositions (i.e., compositions that are suitable for administration to a patient) which can be used in the preparation of unit dosage forms. Such compositions optionally comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of JNK Inhibitor and a second active agent, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which a JNK Inhibitor is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a patient, the pharmaceutically acceptable vehicles are preferably sterile. Water can be the vehicle when the JNK Inhibitor is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propyleneglycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustainedrelease formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In a preferred embodiment, the JNK Inhibitor and optionally the a therapeutic or prophylactic agent are formulated in accordance with routine procedures as pharmaceutical compositions adapted for intravenous administration to human beings. Typically, JNK Inhibitors for intravenous administration are solutions in sterile isotonic aqueous buffer. Where necessary, the compositions can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the JNK Inhibitor is to be administered by infusion, it can be dispensed, for example, with an infusion bottle containing sterile pharmaceutical grade water or saline. Where the JNK Inhibitor is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for an orally administered JNK Inhibitor. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, and the like. Such vehicles are preferably of pharmaceutical grade.

Further, the effect of the JNK Inhibitor can be delayed or prolonged by proper formulation. For example, a slowly soluble pellet of the JNK Inhibitor can be prepared and incorporated in a tablet or capsule. The technique can be improved by making pellets of several different dissolution rates and filling capsules with a mixture of the pellets. Tablets or capsules can be coated with a film which resists dissolution for a predictable period of time. Even the parenteral preparations can be made long-acting, by dissolving or suspending the compound in oily or emulsified vehicles which allow it to disperse only slowly in the serum.

Formulations

Pharmaceutical compositions for use in accordance with the present invention can be formulated in conventional manner using one or more physiologically acceptable vehicles, carriers or excipients.

Thus, the JNK Inhibitor and optionally a second active agent, and their physiologically acceptable salts and solvates, can be formulated into pharmaceutical compositions for administration by inhalation or insufflation (either through the mouth or the nose) or oral, parenteral or mucosol (such as buccal, vaginal, rectal, sublingual) administration. In one embodiment, local or systemic parenteral administration is used.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the JNK Inhibitor.

For buccal administration the pharmaceutical compositions can take the form of tablets or lozenges formulated in conventional manner.

For administration by inhalation, the pharmaceutical compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The pharmaceutical compositions can be formulated for parenteral administration by injection, e.g., by bolus injection or continuous infusion. Formulations for injection can be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The pharmaceutical compositions can also be formulated in rectal compositions such as suppositories or retention enemas, e.g., containing conventional suppository bases such as cocoa butter or other glycerides.

In addition to the formulations described previously, the pharmaceutical compositions can also be formulated as a depot preparation. Such long acting formulations can be administered by implantation (for example subcutaneously or intramuscularly) or by intramuscular injection. Thus, for example, the pharmaceutical compositions can be formulated with suitable polymeric or hydrophobic materials (for example as an emulsion in an acceptable oil) or ion exchange resins, or as sparingly soluble derivatives, for example, as a sparingly soluble salt.

The invention also provides that a pharmaceutical composition can be packaged in a hermetically sealed container such as an ampoule or sachette indicating the quantity. In one embodiment, the pharmaceutical composition is supplied as a dry sterilized lyophilized powder or water free concentrate in a hermetically sealed container and can be reconstituted, e.g., with water or saline to the appropriate concentration for administration to a patient.

The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

In certain preferred embodiments, the pack or dispenser contains one or more unit dosage forms containing no more than the recommended dosage formulation as determined in the Physician 's Desk Reference (₅₆ th ed. 2002, herein incorporated by reference in its entirety).

### Routes of Administration

Methods of administering a JNK Inhibitor and optionally a second active agent include, but are not limited to, parenteral administration (e.g., intradermal, intramuscular, intraperitoneal, intravenous and subcutaneous), topical, epidural, and mucosal (e.g., intranasal, rectal, vaginal, sublingual, buccal or oral routes). In a specific embodiment, the JNK Inhibitor and optionally the second active agent are administered intramuscularly, intravenously, or subcutaneously. The JNK Inhibitor and optionally the second active agent can also be administered by infusion or bolus injection and can be administered together with other biologically active agents. Administration can be local or systemic. The JNK Inhibitor and optionally the second active agent and their physiologically acceptable salts and solvates can also be administered by inhalation or insufflation (either through the mouth or the nose). In one embodiment, local or systemic parenteral administration is used.

In specific embodiments, it can be desirable to administer the JNK Inhibitor locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, said implant being of a porous, nonporous, or gelatinous material, including membranes, such as sialastic membranes, or fibers. In one embodiment, administration can be by direct injection at the site (or former site) of an atherosclerotic plaque tissue. In another embodiment, the JNK Inhibitor can be administered directly to the eye by, for example, an eye dropper.

Pulmonary administration can also be employed, e.g., by use of an inhaler or nebulizer, and formulation with an aerosolizing agent, or via perfusion in a fluorocarbon or synthetic pulmonary surfactant. In certain embodiments, the JNK Inhibitor can be formulated as a suppository, with traditional binders and vehicles such as triglycerides.

In another embodiment, the JNK Inhibitor can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the JNK Inhibitor can be delivered in a controlled release system. In one embodiment, a pump can be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the JNK Inhibitor, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) can be used.

Dosages

The amount of the JNK Inhibitor that is effective in the treatment, prevention and/or management of MD can be determined by standard research techniques. For example, the dosage of the JNK Inhibitor which will be effective in the treatment, prevention and/or management of MD can be determined by administering the JNK Inhibitor to an animal in a model such as, e.g., the animal models known to those skilled in the art. In addition, in vitro assays can optionally be employed to help identify optimal dosage ranges.

Selection of a particular effective dose can be determined (e.g., via clinical trials) by a skilled artisan based upon the consideration of several factors which will be known to one skilled in the art. Such factors include the disease to be treated, prevented and/or managed, the symptoms involved, the patient's body mass, the patient's immune status and other factors known by the skilled artisan.

The precise dose to be employed in the formulation will also depend on the route of administration, and the seriousness of the MD, and should be decided according to the judgment of the practitioner and each patient's circumstances. Effective doses can be extrapolated from dose-response curves derived from in vitro or animal model test systems.

The dose of a JNK Inhibitor to be administered to a patient, such as a human, is rather widely variable and can be subject to independent judgment. It is often practical to administer the daily dose of a JNK Inhibitor at various hours of the day. However, in any given case, the amount of a JNK Inhibitor administered will depend on such factors as the solubility of the active component, the formulation used, patient condition (such as weight), and/or the route of administration.

The general range of effective amounts of the JNK Inhibitor alone or in combination with a second active agent are from about 0.001 mg/day to about 1000 mg/day, more preferably from about 0.001 mg/day to 750 mg/day, more preferably from about 0.001 mg/day to 500 mg/day, more preferably from about 0.001 mg/day to 250 mg/day, more preferably from about 0.001 mg/day to 100 mg/day, more preferably from about 0.001 mg/day to 75 mg/day, more preferably from about 0.001 mg/day to 50 mg/day, more preferably from about 0.001 mg/day to 25 mg/day, more preferably from about 0.001 mg/day to 10 mg/day, more preferably from about 0.001 mg/day to 1 mg/day. Of course, it is often practical to administer the daily dose of compound in portions, at various hours of the day. However, in any given case, the amount of compound administered will depend on such factors as the solubility of the active component, the formulation used, subject condition (such as weight), and/or the route of administration.

Kits

The invention provides a pharmaceutical pack or kit comprising one or more containers containing a JNK Inhibitor and optionally one or more second active agents useful for the treatment, prevention and/or management of MD. The invention also provides a pharmaceutical pack or kit comprising one or more containers containing one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration; or instructions for the composition's use.

The present invention provides kits that can be used in the above methods. In one embodiment, a kit comprises a JNK Inhibitor, in one or more containers, and optionally one or more second active agents useful for the treatment, prevention and/or management of MD, in one or more additional containers.

JNK Inhibitor Activity Assays

The ability of a JNK Inhibitor to inhibit JNK and accordingly, to be useful for the treatment, prevention and/or management of MD, can be demonstrated using one or more of the following assays.

### EXAMPLE: BIOLOGICAL ACTIVITY OF 5-AMINO-ANTHRA(9,1-CD)ISOTHIAZOL-6-ONE

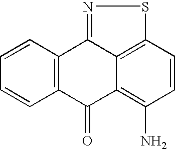

JNK Assay

To 10 μL of 5-amino-anthra(9, 1-cd)isothiazol-6-one in 20% DMSO/80% dilution buffer containing of 20 mM HEPES (pH 7.6), 0.1 mM EDTA, 2.5 mM magnesium chloride, 0.004% Triton ×100, 2 μg/mL leupeptin, 20 mM β-glycerolphosphate, 0.1 mM sodium vanadate, and 2 mM DTT in water was added 30 μL of 50-200 ng His6-JNK1, JNK2, or JNK3 in the same dilution buffer. The mixture was pre-incubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-32P ATP in water was added and the reaction was allowed to proceed for 1 hour at room temperature. The c-Jun phosphorylation was terminated by addition of 150 μL of 12.5% trichloroacetic acid. After 30 minutes, the precipitate was harvested onto a filter plate, diluted with 50 μL of the scintillation fluid and quantified by a counter. The IC₅₀ values were calculated as the concentration of 5-amino-anthra(9, 1-cd)isothiazol-6-one at which the c-Jun phosphorylation was reduced to 50% of the control value. Compounds that inhibit JNK preferably have an IC₅₀ value ranging 0.01-10 μM in this assay. 5-Amino- anthra(9,1-cd)isothiazol-6-one has an IC₅₀ according to this assay of 1 μM for JNK2 and 400 nM for JNK3. The measured IC₅₀ value for 5-amino-anthra(9,1-cd)isothiazol-6-one, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one inhibits JNK. This assay demonstrates that 5-amino-anthra(9,1-cd)isothiazol-6-one, an illustrative JNK Ihibitor, inhibits JNK2 and JNK3 and, accordingly, is useful for the the treatment, prevention and/or management of MD.

Selectivity For JNK:

5-Amino-anthra(9,1-cd)isothiazol-6-one was also assayed for its inhibitory activity against several protein kinases, listed below, using techniques known to those skilled in art (See, e.g., Protein Phosphorylation, Sefton & Hunter, Eds., Academic Press, pp. 97-367, 1998). The following IC₅₀ values were obtained:

| Enzyme | IC₅₀ |
|---|---|
| p38-2 | >30,000 nM |
| MEK6 | >30,000 nM |
| LKK1 | >30,000 nM |
| IKK2 | >30,000 nM |

This assay shows that 5-amino-anthra(9,1-cd)isothiazol-6-one, an illustrative JNK Inhibitor, selectively inhibits JNK relative to other protein kinases and, accordingly, is a selective JNK Inhibitor. Therefore, 5-amino-anthra(9,1-cd)isothiazol-6one, an illustrative JNK Inhibitor, is useful for the the treatment, prevention and/or management of MD.

Jurkat T-cell IL-2 Production Assay:

Jurkat T cells (clone E6-1) were purchased from the American Type Culture Collection of Manassas, Va. and maintained in growth media consisting of RPMI 1640 medium containing 2 mM L-glutamine (commercially available from Mediatech Inc. of Hemdon, Va.), with 10% fetal bovine serum (commercially available from Hyclone Laboratories Inc. of Omaha, Ne.) and penicillin/streptomycin. All cells were cultured at 37° C. in 95% air and 5% CO₂. Cells were plated at a density of 0.2×10⁶ cells per well in 200 IL of media. Compound stock (20 mM) was diluted in growth media and added to each well as a lOx concentrated solution in a volume of 25 μL, mixed, and allowed to pre-incubate with cells for 30 minutes. The compound vehicle (dimethylsulfoxide) was maintained at a final concentration of 0.5% in all samples. After 30 minutes the cells were activated with PMA (phorbol myristate acetate, final concentration 50 ng/mL) and PHA (phytohemagglutinin, final concentration 2 μg/mL). PMA and PHA were added as a 10× concentrated solution made up in growth media and added in a volume of 25 μL per well. Cell plates were cultured for 10 hours. Cells were pelleted by centrifugation and the media removed and stored at −20° C. Media aliquots are analyzed by sandwich ELISA for the presence of IL-2 as per the manufacturers instructions (Endogen Inc. of Woburn, Mass.). The IC₅₀ values were calculated as the concentration of 5-amino-anthra(9,1-cd)isothiazol-6-one at which the IL-2 production was reduced to 50% of the control value. Compounds that inhibit JNK preferably have an IC₅₀ value ranging from 0.1-30 μM in this assay. 5-Amino-anthra(9,1-cd)isothiazol-6-one has an IC₅₀ of 30 μM. The measured IC₅₀ value for 5-amino-anthra(9,1 cd)isothiazol-6-one, as measured by the above assay, however, shows some variability due to the limited solubility of 5-amino-anthra(9,1-cd)isothiazol-6-one in aqueous media. Despite the variability, however, the assay consistently does show that 5-amino-anthra(9,1-cd)isothiazol-6-one inhibits JNK.

This assay shows that 5-amino-anthra(9,1-cd)isothiazol-6-one, an illustrative JNK Inhibitor, inhibits IL-2 production in Jurkat T-cells and accordingly inhibits JNK. Therefore, 5-amino-anthra(9,1-cd)isothiazol-6-one, an illustrative JNK Inhibitor, is useful for the the treatment, prevention and/or management of MD.

[³H]Dopamine Cell Culture Assay:

Cultures of dopaminergic neurons were prepared according to a modification of the procedure described by Raymon and Leslie (J. Neurochem. 62:1015-1024, 1994). Time-mated pregnant rats were sacrificed on embyronic day 14-15 (crown rump length 11-12 mm) and the embryos removed by cesarean section. The ventral mesencephalon, containing the dopaminergic neurons, was dissected from each embryo. Tissue pieces from approximately 48 embryos were pooled and dissociated both enzymatically and mechanically. An aliquot from the resulting cell suspension was counted and the cells were plated in high glucose DMEM/F12 culture medium with 10% fetal bovine serum at a density of 1×10⁵ cells/well of a Biocoat poly-D-lysine-coated 96-well plate. The day following plating was considered 1 day in vitro (DIV). Cells were maintained in a stable environment at 37° C., 95% humidity, and 5% CO₂. A partial medium change was performed at 3 DIV. At 7 DIV, cells were treated with the neurotoxin, 6-hydroxydopamine (6—OHDA, 30 μM) in the presence and absence of 5-amino-anthra(9,1-cd)isothiazol-6-one. Cultures were processed for [³H]dopamine uptake 22 hours later.

[³H]Dopamine uptake is used as a measure of the health and integrity of dopaminergic neurons in culture (Prochiantz et al., PNAS 76: 5387-5391, 1979). It was used in these studies to monitor the viability of dopaminergic neurons following exposure to the neurotoxin 6-OHDA. 6-OHDA has been shown to damage dopaminergic neurons both in vitro and in vivo and is used to model the cell death observed in Parkinson's disease (Ungerstedt, U., Eur. J. Pharm., 5 (1968) 107-110 and Hefti et al., Brain Res., 195 (1980) 123-137). Briefly, cells treated with 6-OHDA in the presence and absence of 5-amino-anthra(9, 1-cd)isothiazol-6-one were assessed in the uptake assay 22 hrs after exposure to 6-OHDA. Culture medium was removed and replaced with warm phosphate buffered saline (PBS) with calcium and magnesium, 10 μM pargyline, 1 mM ascorbic acid, and 50 nM [³H]dopamnine. Cultures were incubated at 37° C. for 20 min. Rₐdioactivity was removed and the cultures were washed 3x with ice cold PBS. To determine the intracellular accumulation of [³H]dopamine, cells were lysed with M-PER detergent and an aliquot was taken for liquid scintillation counting. The measured effect of 5-amino-anthra(9,1-cd) isothiazol-6-one on the intracellular accumulation of [³H]dopamine, as measured by the above assay, however, shows some variability due to the limited solubility of 5-amino-anthra(9,1-cd)isothiazol-6-one in aqueous media. Despite the variability, however, the assay consistently does show that 5-amino-anthra(9, 1-cd)isothiazol-6-one protects rat ventral mesencephalan neurons from the toxic effects of 6-OHDA. Accordingly, 5-amino-anthra(9,1-cd)isothiazol-6-one, an illustrative JNK Inhibitor, is useful for the the treatment, prevention and/or management of MD.

Brain-Blood Plasma Distribution of 5-amino-anthra(9,1-cd)isothiazol-6-one In Vivo

5-Amino-anthra(9,1-cd)isothiazol-6-one was administered intravenously (10 mg/kg) into the veins of Sprague-Dawley rats. After 2 hr, blood samples were obtained from the animals and their vascular systems were perfused with approximately 100 mL of saline to rid their brains of blood. The brains were removed from the animals, weighed, and homogenized in a 50 mL conical tube containing 10 equivalents (w/v) of methanol/saline (1:1) using a Tissue Tearer (Fischer Scientific). The homogenized material was extracted by adding 600 μL of cold methanol to 250 μL of brain homogenate vortexed for 30 sec and subjected to centrifugation for 5 min. After centrifugation, 600 μL of the resulting supernatant was transferred to a clean tube and evaporated at room temperature under reduced pressure to provide a pellet. The resulting pellet was reconstituted in 250 μL of 30% aqueous methanol to provide a brain homogenate analysis sample. A plasma analysis sample was obtained using the brain homogenate analysis sample procedure described above by substituting plasma for brain homogenate. Standard plasma samples and standard brain homogenate samples containing known amounts of 5-amino-anthra(9,1-cd)isothiazol-6-one were also prepared by adding 5 μLL of serial dilutions (50:1) of a solution of 5-amino-anthra(9,1-cd)isothiazol-6-one freshly prepared in cold ethanol to 250 μLL of control rat plasma (Bioreclamation of Hicksville, N.Y.) or control brain homogenate. The standard plasma samples and standard brain homogenate samples were then subjected to the same extraction by protein precipitation, centrifugation, evaporation, and reconstitution procedure used for the brain homogenate to provide brain homogenate standard analysis samples and plasma standard analysis samples. The brain homogenate analysis samples, plasma analysis samples, and standard analysis samples were analyzed and compared using HPLC by injecting 100 μL of a sample onto a 5 μm C-18 Luna column (4.6 mm×150 mm, commercially available from Phenomenex of Torrance, Calif.) and eluting at 1 mL/min with a linear gradient of 30% aqueous acetonitrile containing 0.1% trifluoroacetic acid to 90% aqueous acetonitrile containing 0.1% trifluoroacetic acid over 8 minutes and holding at 90% aqueous acetonitrile containing 0.1 % trifluoroacetic acid for 3 min. with absorbance detection at 450 nm. Recovery of 5-amino-anthra(9,1-cd)isothiazol-6-one was 56-5.7% for plasma and 42±6.2% for the brain. The concentration of 5-amino-anthra(9,1-cd) isothiazol-6-one in the brain and plasma was determined by comparing HPLC chromatograms obtained from the brain homogenate analysis samples and plasma analysis samples to standard curves constructed from analysis of the brain homogenate standard analysis samples and the plasma standard analysis samples, respectively. Results from this study show that 5-amino-anthra(9,1-cd)isothiazol-6-one, following intravenous administration, crosses the blood-brain barrier to a significant extent. In particular, brain-drug concentrations were approximately 65 nmole/g and plasma concentrations were approximately 7 μM at 2 hr post-dose, resulting in a brain-plasma concentration ratio of approximately 9-fold (assuming 1 g of brain tissue is equivalent to 1 mL of plasma). This example shows that 5-amino-anthra(9,1-cd)isothiazol-6-one, an illustrative JNK Inhibitor, has enhanced ability to cross the blood-brain barrier. In addition, this example shows that the JNK Inhibitors, in particular 5-amino-anthra(9,1-cd)isothiazol-6-one, can cross the blood-brain barrier when administered to a patient.

Macular Degeneration Clinical Study

Forty patients with macular degeneration are divided into two groups. The first group receives conventional treatment for closing the leaking choroidal vessels (characteristic of this disease) by photodynamic therapy with verteporfin (see for example Ophthalmol. 117:1329-1345 (1999). The second group receives the same conventional therapy with verteporfin with 1-(5-(1H-1,2,4-triazol-5-yl)(1H-indazol-3-yl))-3-(2-piperidylethoxy)benzene at about 300 mg/day as an adjuvant for 20 weeks.

The neovascular cascade is sufficiently hindered in the group receiving 1-(5-(1H-1,2,4-triazol-5-yl)( 1 H-indazol-3-yl))-3-(2-piperidylethoxy)benzene to indefinitely prolong the effects of the photodynamic therapy. The first group, without 1-(5-(1H 1,2,4-triazol-5-yl)( 1 H-indazol-3-yl))-3-(2-piperidylethoxy)benzene, however will experience progressive reperfusion of the ablated vessels several weeks after treatment. Progressive visual loss follows which requires the photodynamic therapy to be repeated.

It is understood that other preferred embodiments are when 1-(5-(1 H-1,2,4-triazol-5-yl)(1H-indazol-3-yl))-3-(2-piperidylethoxy)benzene is administered at about 75-900 mgs/day or a greater dose, generally about 1.5 to 2.5 times the daily dose every other day. It is further understood that the adjuvant therapy is applicable to other types of conventional therapy used to treat or prevent MD such as, but not limited to surgical intervention including laser photocoagulation.

It will be appreciated that, although specific embodiments of the invention have been described herein for purposes of illustration, the invention described and claimed herein is not to be limited in scope by the specific embodiments herein disclosed. These embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

A number of references have been cited, the entire disclosure of which are incorporated herein by reference in their entirety.

What is claimed is:

1. A method for treating or preventing MD in a patient, comprising administering to a patient in need thereof an effective amount of a JNK Inhibitor or a pharmaceutically acceptable salt, solvate or stereoisomer thereof.

2. A method for treating or preventing MD in a patient, comprising administering to a patient in need thereof an effective amount of a compound having the following formula: 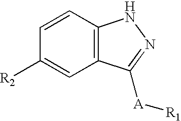
or a pharmaceutically acceptable salt, solvate or stereoisomer thereof, wherein:
A is a direct bond, —(CH₂)ₐ—, —(CH₂)_{b}CH═CH(CH₂)_{c}—, or —(CH₂)_{b}C|C(CH₂)_{c}—;
R₁ is aryl, heteroaryl or heterocycle fused to phenyl, each being optionally substituted with one to four substituents independently from R₃;
R₂ is —R₃, —R₄, —(CH₂)_{b}C(═O)R₅, —(CH₂)_{b}C(═O)OR₅) —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅,—(CH₂)_{b}SO_{d}R₅ or —(CH₂)_{b}SO₂NR₅R₆;
a is 1, 2, 3, 4, 5 or 6;
b and c are the same or different and at each occurrence independently 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, substituted aryl, arylalkyl, heterocycle, heterocycloalkyl, —C(═O)OR₈, —OC(═O)R₈, —C(═O)NR₈R₉, —C(═O)NR₈OR₉, —SO₂NR₉, —NR₈SO₂R₉, —CN, —NO₂, —NR₈R₉, —NR₉C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independentlyfrom R₃; and R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to formn a heterocycle are optionally substituted with one to four substituents independently from R₃.

3. A method for treating or preventing MD in a patient, comprising administering to a patient in need thereof an effective amount of a compound having the following formula: 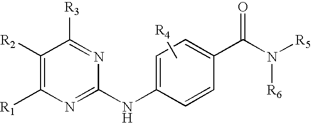
or a pharmaceutically acceptable salt, solvate or stereoisomer thereof, wherein:
R₁ is aryl or heteroaryl optionally substituted with one to four substituents independently from R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNRiI C(═O)NR₉R₁₀, (CH₂)ₐNR₉R₁₀, —(CH₂)ₐOR₉, —(CH₂)ₐ SO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₉, —C(═O)NR₈OR₉, —SO,R₈, —SO_{c}NR₈R₉, —NR₈SOCR₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀and R₁₁ are the same or different and at each occurrence independently hydrogen, alkyl, substituted alkyl, aryl, arylalkyl, heterocycle or heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

4. A method for treating or preventing MD in a patient, comprising administering to a patient in need thereof an effective amount of a compound having the following formula: 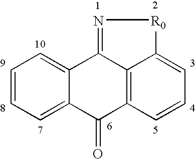
or a pharmaceutically acceptable salt, solvate or stereoisomer thereof,
wherein R₀ is —O—, —S—, —S(O)—, —S(O)₂—, NH or —CH₂—;
the compound being (i) unsubstituted, (ii) monosubstituted and having a first substituent, or (iii) disubstituted and having a first substituent and a second substituent;
the first or second substituent, when present, is at the 3, 4, 5, 7, 8, 9, or 10 position, wherein the first and second substituent, when present, are independently alkyl, hydroxy, halogen, nitro, trifluoromethyl, sulfonyl, carboxyl, alkoxycarbonyl, alkoxy, aryl, aryloxy, arylalkyloxy, arylalkyl, cycloalkylalkyloxy, cycloalkyloxy, alkoxyalkyl, alkoxyalkoxy, aminoalkoxy, mono-alkylaminoalkoxy, di-alkylaminoalkoxy, or a group represented by formula (a), (b), (c), (d), (e), or (f): 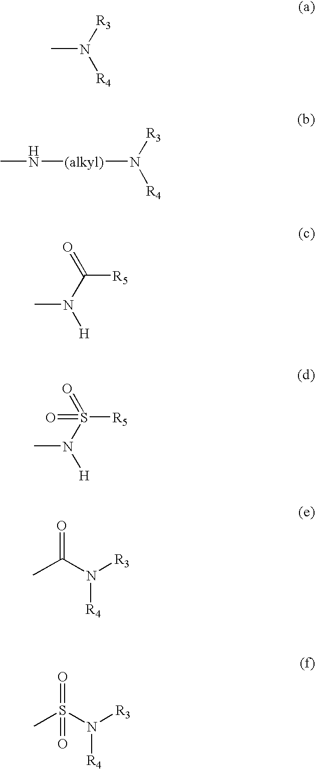
wherein R₃ and R₄ are taken together and represent alkylidene or a heteroatomcontaining cyclic alkylidene or R₃ and R₄ are independently hydrogen, alkyl, cycloalkyl, aryl, arylalkyl, cycloalkylalkyl, aryloxyalkyl, alkoxyalkyl, aminoalkyl, monoalkylaminoalkyl, or di-alkylaminoalkyl; and
R₅ is hydrogen, alkyl, cycloalkyl, aryl, arylalkyl, cycloalkylalkyl, alkoxy, alkoxyalkyl, alkoxycarbonylalkyl, amino, mono-alkylamino, di-alkylamino, arylamino, arylalkylaamino, cycloalkylamino, cycloalkylalkylamino, aminoalkyl, monoalkylaminoalkyl, or di-alkylaminoalkyl.

5. The method of claim 2 wherein A is a direct bond.

6. The method of claim 2 wherein A is —(CH₂)ₐ—.

7. The method of claim 2 wherein A is —(CH₂)_{b}CH═CH(CH₂)_{c}—.

8. The method of claim 2 wherein A is —(CH₂)_{b}C|C(CH₂)_{c}—.

9. The method of claim 2 wherein the compound has the following formula: 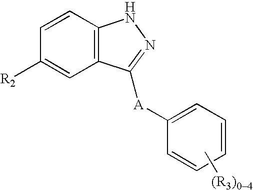
or a pharmaceutically acceptable salt, solvate or stereoisomer thereof, wherein:
A is a direct bond, —(CH₂)ₐ—, —(CH₂)_{b}CH═CH(CH₂)_{c}—, or —(CH₂)_{b}C|C(CH₂)_{c}—;
R₁ is aryl, heteroaryl or heterocycle fused to phenyl, each being optionally substituted with one to four substituents independently from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₅OR₉, —SO₂NR₈R₉, —NR₈SO₂R₉, —CN, —NO₂, —NR₈R₉, —NR₈C(═O)R₉, —NC(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently from R₃.

10. The method of claim 2 wherein the compound has the following formula: 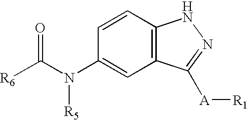
or a pharmaceutically acceptable salt, solvate or stereoisomer thereof, wherein:
A is a direct bond, —(CH₂)ₐ—, —(CH₂)_{b}CH═CH(CH₂)_{c}—, or —(CH₂)_{b}C C(CH₂)_{c}—;
R₁ is aryl, heteroaryl or heterocycle fused to phenyl, each being optionally substituted with one to four substituents independently from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
a is 1, 2, 3, 4, 5 or 6;
b and c are the same or different and at each occurrence independently 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently from R₃.

11. The method of claim 2 wherein the compound has the following formula: 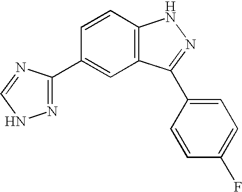
or a pharmaceutically acceptable salt, solvate or stereoisomer thereof.

12. The method of claim 3, wherein the compound has the following formula: 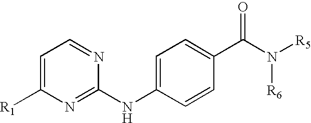
or a pharmaceutically acceptable salt, solvate or stereoisomer thereof, wherein:
R₁ is aryl or heteroaryl optionally substituted with one to four substituents independently from R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNR₁C(═O)NR₉R₁₀, (CH₂)ₐNR₉R₁₀, —(CH₂)ₐOR₉, —(CH₂)ₐSO_{c}R₉ or —(CH₂)ₐSO₂NR₉R₁₀;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO_{c}R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀and R₁₁ are the same or different and at each occurrence independently hydrogen, alkyl, substituted alkyl, aryl, substituted aryl, arylalkyl, heterocycle, heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

13. The method of claim 3, wherein the compound has the following formula: 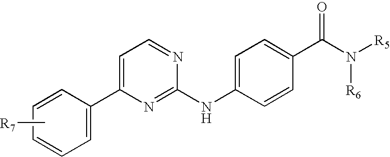
or a pharmaceutically acceptable salt, solvate or stereoisomer thereof, wherein:
R₁ is aryl or heteroaryl optionally substituted with one to four substituents independently from R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)ₐC(═O)R₉, —(CH₂)ₐC(═O)OR₉, —(CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)ₐNRiI C(═O)NR₉R₁₀, —(CH₂)ₐNR₉R₁₀, —(CH₂)ₐOR₉, —(CH₂)ₐSO_{c}R₉ or —(CH₂)ₐSO₂NR₉Rio;
or R₅ and R₆ taken together with the nitrogen atom to which they are attached to form a heterocycle or substituted heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SOR₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, aryl, arylalkyl, heterocycle, heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

14. The method of claim 3, wherein the compound has the following formula: 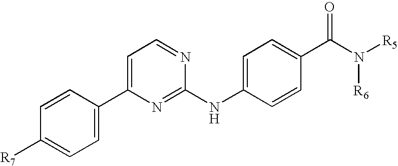
or a pharmaceutically acceptable salt, solvate or stereoisomer thereof, wherein:
R₁ is aryl or heteroaryl optionally substituted with one to four substituents independently from R₇;
R₂ is hydrogen;
R₃ is hydrogen or lower alkyl;
R₄ represents one to four optional substituents, wherein each substituent is the same or different and independently halogen, hydroxy, lower alkyl or lower alkoxy;
R₅ and R₆ are the same or different and independently —R₈, —(CH₂)₀C(═O)R₉, —(CH₂)ₐC(═O)OR₉, (CH₂)ₐC(═O)NR₉R₁₀, —(CH₂)ₐC(═O)NR₉(CH₂)_{b}C(═O)R₁₀, —(CH₂)ₐNR₉C(═O)R₁₀, (CH₂)₀NRIIC(═O)NR₉R₁₀, —(CH₂)ₐNR₉R₁₀, —(CH₂)ₐOR₉, —(CH₂)₀SO_{c}R₉ or —(CH₂)ₐSO₂NR₉R₁₀;
or R₅ and R₆ taken together with the nitrogen atom to which they are attached to form a heterocycle;
R₇ is at each occurrence independently halogen, hydroxy, cyano, nitro, carboxy, alkyl, alkoxy, haloalkyl, acyloxy, thioalkyl, sulfinylalkyl, sulfonylalkyl, hydroxyalkyl, aryl, arylalkyl, heterocycle, heterocycloalkyl, —C(═O)OR₈, —OC(═O)R₈, —C(═O)NR₈R₉, —C(═O)NR₈OR₉, —SO_{c}R₈, —SO_{c}NR₈R₉, —NR₈SO,R₉, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₈, R₉, R₁₀ and R₁₁ are the same or different and at each occurrence independently hydrogen, alkyl, substituted alkyl, aryl, arylalkyl, heterocycle, heterocycloalkyl;
or R₈ and R₉ taken together with the atom or atoms to which they are attached to form a heterocycle;
a and b are the same or different and at each occurrence independently 0, 1, 2, 3 or 4; and
c is at each occurrence 0, 1 or 2.

15. The method of claim 4, wherein R₀ is —O—.

16. The method of claim 4, wherein R₀ is —S—.

17. The method of claim 4, wherein R₀ is —S(O)—.

18. The method of claim 4, wherein R₀ is —S(O)₂—.

19. The method of claim 4, wherein R₀ is NH.

20. The method of claim 4, wherein R₀ is CH₂—.

21. The method of claim 4, wherein the compound has the following formula: 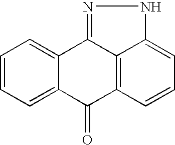
or a pharmaceutically acceptable salt, solvate or stereoisomer thereof.

22. The method of claim 1, further comprising administering an effective amount of a steroid, a light sensitizer, an integrin, an antioxidant, an interferon, a xanthine derivative, a growth hormone, a neutrotrophic factor, a regulator of neovascularization, an antiVEGF antibody, a prostaglandin, an antibiotic, a phytoestrogen, an anti-inflammatory compound, an IMiD@, a SelCID@, an antiangiogenesis compound, or a combination thereof.

23. The method of claim 2, further comprising administering an effective amount of a steroid, a light sensitizer, an integrin, an antioxidant, an interferon, a xanthine derivative, a growth hormone, a neutrotrophic factor, a regulator of neovascularization, an antiVEGF antibody, a prostaglandin, an antibiotic, a phytoestrogen, an anti-inflammatory compound, an IMiD®, a SelCID®, an antiangiogenesis compound, or a combination thereof.

24. The method of claim 3, further comprising administering an effective amount of a steroid, a light sensitizer, an integrin, an antioxidant, an interferon, a xanthine derivative, a growth hormone, a neutrotrophic factor, a regulator of neovascularization, an antiVEGF antibody, a prostaglandin, an antibiotic, a phytoestrogen, an anti-inflammatory compound, an IMiD@, a SelCID@, an antiangiogenesis compound, or a combination thereof.

25. The method of claim 4, further comprising administering an effective amount of a steroid, a light sensitizer, an integrin, an antioxidant, an interferon, a xanthine derivative, a growth hormone, a neutrotrophic factor, a regulator of neovascularization, an antiVEGF antibody, a prostaglandin, an antibiotic, a phytoestrogen, an anti-inflammatory compound, an IMiD@, a SelCID@, an antiangiogenesis compound, or a combination thereof.

26. The method of claim 1, wherein the MD is wet MD.

27. The method of claim 1, wherein the MD is dry MD.

28. The method of claim 1, further comprising the administration of verteporfin.

29. The method of claim 22, wherein antiangiogenesis compound is thalidomide.

30. The method of claim 22, wherein the anti-VEGF antibody is rhuFab.

31. The method of claim 22, wherein the the xanthine derivative is pentoxifylline.

32. The method of claim 22, wherein the interferon is interferon-2a.

33. The method of claim 1, further comprising administering laser photocoagulation therapy.

34. The method of claim 1 further comprising administering photodynamic therapy.

35. A method for treating or preventing ARM, CNVM, PED or atrophy of RPE, which comprises administering to a patient in need of such treatment or prevention an effective amount of a JNK inhibitor or a pharmaceutically acceptable, salt, solvate or stereoisomer thereof.

36. The method of claim 35, further comprising administering an effective amount of a steroid, a light sensitizer, an integrin, an antioxidant, an interferon, a xanthine derivative, a growth hormone, a neutrotrophic factor, a regulator of neovascularization, an anti-VEGF antibody, a prostaglandin, an antibiotic, a phytoestrogen, an anti-inflammatory compound or an antiangiogenesis compound.

37. A pharmaceutical composition comprising an effective amount of a JNK Inhibitor and a steroid, a light sensitizer, an integrin, an antioxidant, an interferon, a xanthine derivative, a growth hormone, a neutrotrophic factor, a regulator of neovascularization, an anti-VEGF antibody, a prostaglandin, an antibiotic, a phytoestrogen, an antiangiogenesis compound, or a combination thereof.
